(19) United States
(12) Patent Application Publication
(10) Pub. No.: US 2022/0361731 A1
(43) Pub. Date: Nov. 17, 2022

(54) LIGHT DISTRIBUTION INSPECTION DEVICE, LIGHT DISTRIBUTION INSPECTION METHOD, ENDOSCOPE SYSTEM, AND STORAGE MEDIUM

(71) Applicant: OLYMPUS CORPORATION, Tokyo (JP)

(72) Inventors: Hiroyuki KAMEE, Tokyo (JP); Yasuo SASAKI, Tokyo (JP)

(73) Assignee: OLYMPUS CORPORATION, Tokyo (JP)

(21) Appl. No.: 17/875,575

(22) Filed: Jul. 28, 2022

Related U.S. Application Data
(63) Continuation of application No. PCT/JP2020/003182, filed on Jan. 29, 2020.

(51) Int. Cl. A61B 1/00; A61B 1/06; A61B 1/07

(52) U.S. Cl. CPC A61B 1/00009; A61B 1/0655; A61B 1/0638; A61B 1/07

(57) ABSTRACT

Provided is a light distribution inspection device capable of accurately and easily inspecting light quantity and luminous intensity distribution of light emitted from a light guide member. A light distribution inspection device comprising a processor, wherein the processor is configured to: acquire, based on a plurality of beams of inspection light each incident on a light guide of an illumination device, a light quantity distribution characteristic of each of a plurality of beams of emission light emitted from each of a plurality of emission sections of the illumination device optically connected to the light guide, in association with incident position information of each of the beams of inspection light; calculate light distribution information of the illumination device, based on each of pieces of the incident position information and each of the light quantity distribution characteristics; and analyze the light distribution information to calculate light quantity and luminous intensity distribution.

### CROSS-REFERENCE TO RELATED APPLICATION

The present application is a continuation application of PCT/JP2020/003182 filed on Jan. 29, 2020; the entire contents of which are incorporated herein by reference.

### BACKGROUND

### Technical Field

The present disclosure relates to a light distribution inspection device, a light distribution inspection method, an endoscope system, and a storage medium.

### Description of the Related Art

WO 2018/235166 discloses an endoscope system. The endoscope system includes a scope and an illumination light supply device. The scope includes an image pickup unit, a light guide, and an illumination light emission unit. The illumination light supply device includes a light source unit and a light quantity distribution changing device.

The light quantity distribution changing device transmits illumination light emitted from the light source unit to the light guide. The illumination light emitted from the light source unit is changed by the light quantity distribution changing device so that the light quantity distribution of the illumination light emitted from the illumination light emission unit becomes a desired light quantity distribution. With this change, the light quantity distribution of illumination light within an illumination light irradiation area irradiated with the illumination light is changed.

### SUMMARY

A light distribution inspection device according to at least some embodiments of the present disclosure includes a processor.

The processor is configured to:

acquire, based on a plurality of beams of inspection light each incident on a light guide of an illumination device, a light quantity distribution characteristic of each of a plurality of beams of emission light emitted from each of a plurality of emission sections of the illumination device optically connected to the light guide, in association with incident position information of each of the beams of inspection light;

calculate light distribution information of the illumination device, based on each of pieces of the incident position information and each of the light quantity distribution characteristics; and

analyze the light distribution information to calculate light quantity and luminous intensity distribution.

A light distribution inspection method according to at least some embodiments of the present disclosure includes:

generating inspection light from illumination light having a beam diameter including an incident surface of a light guide,

the inspection light being incident light incident on the light guide;

generating an image based on the inspection light in association with incident position information on an incident position of the inspection light;

calculating light distribution information based on the image based on the inspection light and the incident position information; and

analyzing the light distribution information to calculate light quantity and luminous intensity distribution,

the light distribution information being information on a light distribution characteristic of the inspection light emitted from the light guide.

An endoscope system according to at least some embodiments of the present disclosure includes:

a light source device including a light source and an illumination controller configured to control emission light from the light source; and

an endoscope including a light guide member connectable to the light source device and having a light guide, an image pickup unit configured to acquire an image, a memory configured to store therein light distribution information of illumination light generated based on the emission light, and a plurality of emission sections optically connected to the light guide and each configured to emit a plurality of beams of illumination light based on the emission light, and

the illumination controller controls the emission light based on the light distribution information acquired from the memory to control light distribution of the illumination light emitted from at least one of the emission sections.

A storage medium according to at least some embodiments of the present disclosure stores therein a program to cause processing comprising:

generating inspection light from illumination light having a beam diameter including an incident surface of a light guide,

the inspection light being incident light incident on the light guide;

generating an image based on the inspection light in association with incident position information on an incident position of the inspection light;

calculating light distribution information based on the image based on the inspection light and the incident position information; and

analyzing the light distribution information to calculate light quantity and luminous intensity distribution,

the light distribution information being information on a light distribution characteristic of the inspection light emitted from the light guide.

An endoscope system according to at least some embodiments of the present disclosure is an endoscope system including:

an endoscope; and

a light source device, in which

the light source device includes
a light source and
an illumination controller configured to control emission light from the light source,

the endoscope includes
a light guide member having an incident surface on which the emission light controlled is incident,
an emission section optically connected to the light guide member and emitting light guided by the light guide member as illumination light, and
a memory configured to store therein incident position information that is a position at which the emission light is incident on the incident surface, in association with light quantity and luminous intensity distribution of the illumination light emitted from the emission section, and

the illumination controller controls the emission light based on the incident position information, the light quantity, and the luminous intensity distribution stored in the memory.

### BRIEF DESCRIPTION OF THE DRAWINGS

FIG. 1A and FIG. 1B are schematic diagrams of a light distribution inspection device;

FIG. 2 is a diagram illustrating a light distribution inspection device;

FIG. 3A and FIG. 3B are diagrams illustrating a state of illumination light;

FIG. 4A, FIG. 4B, and FIG. 4C are diagrams illustrating a digital mirror device;

FIG. 5A and FIG. 5B are diagrams illustrating a state in which illumination light is reflected;

FIG. 6A, FIG. 6B, and FIG. 6C are diagrams illustrating the digital mirror device and inspection light;

FIG. 7 is a diagram illustrating inspection light emitted from a light guide member;

FIG. 8A and FIG. 8B are diagrams illustrating another light guide member;

FIG. 9A and FIG. 9B are diagrams illustrating inspection light emitted from the light guide member;

FIG. 10 is a diagram illustrating image pickup by a first method;

FIG. 11A and FIG. 11B are diagrams illustrating a field of view of an objective lens, an illumination region, and an image pickup region;

FIG. 12A, FIG. 12B, and FIG. 12C are diagrams illustrating movement of an imager;

FIG. 13 is a diagram illustrating image pickup by a second method;

FIG. 14A, FIG. 14B, and FIG. 14C are diagrams illustrating light selected by the digital mirror device;

FIG. 15 is a diagram illustrating a light distribution inspection device;

FIG. 16A and FIG. 16B are diagrams illustrating a distal end of an endoscope;

FIG. 17A and FIG. 17B are diagrams illustrating a reflector in a first example;

FIG. 18A, FIG. 18B, FIG. 18c and FIG. 18D are diagrams illustrating the reflector in the first example and an image of inspection light;

FIG. 19 are diagrams illustrating a reflector in a second example;

FIG. 20 is a diagram illustrating a reflector in a third example;

FIG. 21A and FIG. 21B are diagrams illustrating reflectors in a fourth example;

FIG. 22A, FIG. 22B, FIG. 22C, and FIG. 22D are diagrams illustrating reflectors in a fifth example;

FIG. 23 is a flowchart of a first light distribution inspection method;

FIG. 24 is a flowchart of a second light distribution inspection method;

FIG. 25 is a diagram illustrating an endoscope system in a first example;

FIG. 26 is a diagram illustrating an endoscope system in a second example;

FIG. 27A, FIG. 27B, FIG. 27C, and FIG. 27D are diagrams illustrating an image pickup state and an image;

FIG. 28 is a diagram illustrating an endoscope system in a third example;

FIG. 29 is a diagram illustrating an endoscope system in a fourth example; and

FIG. 30A and FIG. 30B are diagrams illustrating a reflector in a sixth example.

### DETAILED DESCRIPTION

Prior to a description of examples, operation effects of embodiments according to some aspects of the present disclosure will be described. In a specific description of operation effects of the embodiments, specific examples will be described. However, the examples described later as well as the illustrative aspects are only some of the aspects encompassed by the present disclosure, and these aspects include numerous variations. Therefore, the present disclosure is not intended to be limited to the illustrative aspects.

A light distribution inspection device of the present embodiment includes: an information acquisition unit configured to acquire, based on a plurality of beams of inspection light each incident on a light guide of an illumination device, a light quantity distribution characteristic of each of a plurality of beams of emission light emitted from each of a plurality of emission sections of the illumination device optically connected to the light guide, in association with incident position information of each of the beams of inspection light; and a light distribution information calculating unit configured to calculate light distribution information of the illumination device, based on each of pieces of the incident position information and each of the light quantity distribution characteristics.

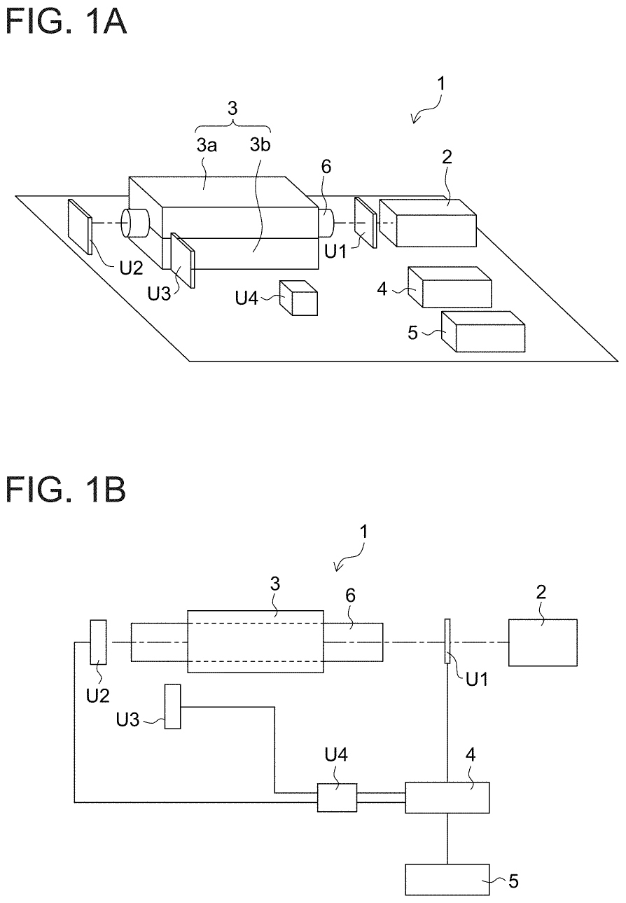

FIG. 1A and FIG. 1B are schematic diagrams of a light distribution inspection device. FIG. 1A is a perspective view of the device. FIG. 1B is a top view.

A light distribution inspection device 1 includes an information acquisition unit 4 and a light distribution information calculating unit 5.

In the light distribution inspection device 1, an illumination device 2 is placed for inspection. The illumination device 2 includes a light guide member 6. For ease of explanation, in FIG. 1A and FIG. 1B, the light guide member 6 separated from the illumination device 2 is depicted.

A holding member 3 includes a first member 3a and a second member 3b. The light guide member 6 is sandwiched between the first member 3a and the second member 3b. The light guide member 6 is held by the holding member 3.

The light guide member 6 includes a light guide. When the light guide member 6 is contained in the illumination device 2, the illumination device 2 includes a light guide. The light guide member 6 includes an emission section. When the light guide member 6 is contained in the illumination device 2, the illumination device 2 includes an emission section. The emission section is optically connected to the light guide.

Inspection light is used in the light distribution inspection device 1. The inspection light is incident light that is incident on the light guide.

In inspection, the incident position of inspection light relative to an incident surface changes over time. Therefore, a plurality of beams of inspection light are incident on the light guide. Incident position information is acquired for each of the beams of inspection light.

The inspection light incident on the incident surface of the light guide is emitted from an emission surface of the light guide. Since there are a plurality of beams of inspection light, a plurality of beams of emission light are emitted from the emission surface of the light guide. Since the emission surface is located at the emission section of the illumination device 2, the beams of emission light are emitted from the emission section of the illumination device 2. Thus, the light quantity distribution characteristic is acquired for each of the beams of emission light.

As a result, a plurality of pieces of incident position information and a plurality of light quantity distribution characteristics are acquired. The information acquisition unit 4 acquires each of the light quantity distribution characteristics in association with each of pieces of the incident position information.

The light quantity distribution characteristic is acquired based on the emission light emitted from the emission section of the illumination device 2. The emission light is generated from inspection light incident on the light guide of the illumination device 2. Thus, the light quantity distribution characteristic is acquired based on the inspection light incident on the light guide.

The light distribution information calculating unit 5 calculates light distribution information of the illumination device 2. The calculation of the light distribution information is performed based on each piece of the incident position information and each light quantity distribution characteristic.

In the light guide member 6, the number of emission sections is one. The light guide member 6 may have a plurality of emission sections. In this case, the beams of emission light are emitted from the emission sections of the illumination device 2, respectively.

It is preferable that the light distribution inspection device 1 include a unit U2 and a unit U3. However, the light distribution inspection device 1 may include either one of the unit U2 or the unit U3. A unit U1, the unit U2, the unit U3, and a unit U4 will be described later.

In the light distribution inspection device of the present embodiment, it is preferable that the inspection light be part of illumination light, an irradiation region is a region irradiated with the inspection light, the irradiation region is narrower than the incident surface of the light guide, the incident position information include information on a position of the irradiation region, and acquisition of the light quantity distribution characteristic and calculation of the light distribution information be performed while a position of the irradiation region is changed. Further, it is preferable that changing a position of the irradiation region be performed by a digital mirror device.

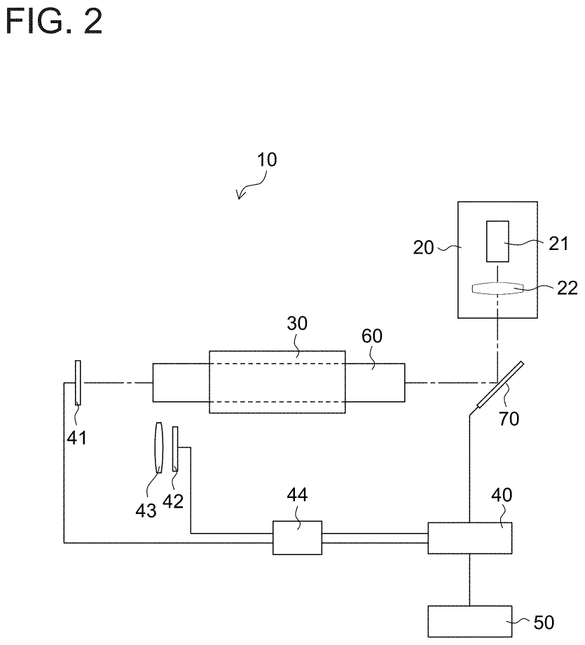

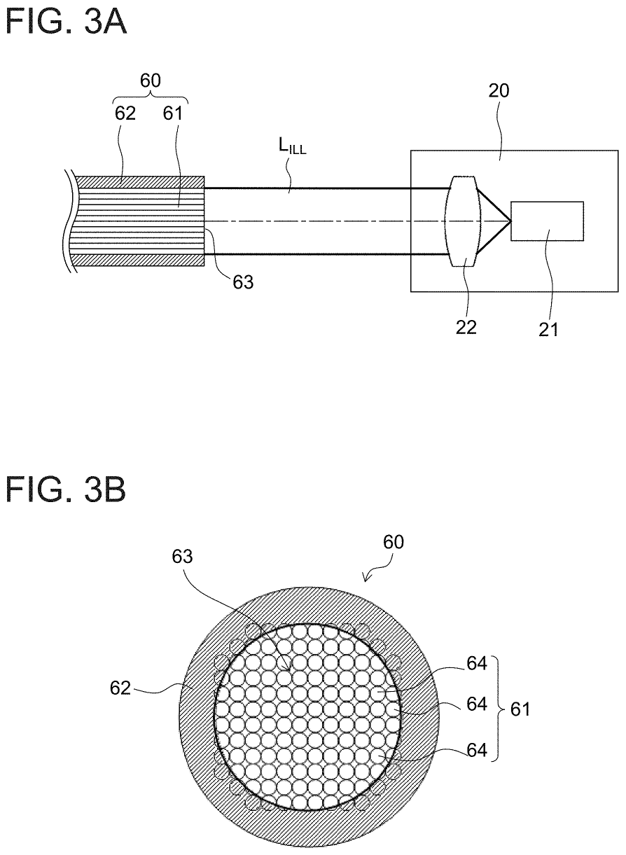

FIG. 2 is a diagram illustrating the light distribution inspection device. FIG. 3A and FIG. 3B are diagrams illustrating a state of illumination light. FIG. 3A is a diagram illustrating illumination light incident on the light guide member. FIG. 3B is a diagram illustrating an end surface of the light guide member.

A light distribution inspection device 10 includes an information acquisition unit 40 and a light distribution information calculating unit 50.

In the light distribution inspection device 10, an illumination device 20 is placed for inspection. The illumination device 20 includes a light source 21, a lens 22, and a light guide member 60. For ease of explanation, in FIG. 2, the light guide member 60 separated from the illumination device 20 is depicted. The light guide member 60 is held by a holding member 30.

FIG. 2 is a schematic diagram. Therefore, an imager 42 and a lens 43 are proximate to each other. In an actual device, the distance between the imager 42 and the lens 43 is appropriately set so that an optical image can be picked up by the imager 42.

The illumination device 20 includes the light source 21 and the lens 22. It is possible to use a laser or an LED as the light source 21. Instead of the lens 22, a plurality of lenses may be used.

The light distribution inspection device 10 includes a digital mirror device 70 (hereinafter referred to as “DMD 70”). The DMD 70 corresponds to the unit U1.

Further, the light distribution inspection device 10 includes an imager 41, the imager 42, the lens 43, and an image generating unit 44. The imager 41 corresponds to the unit U2. The imager 42 and the lens 43 correspond to the unit U3. The image generating unit 44 corresponds to the unit U4.

The imager 41 and the imager 42 output signals to be used to generate images to the image generating unit 44. In FIG. 2, the image generating unit 44 is separated from the information acquisition unit 40. However, the image generating unit 44 may be contained in the information acquisition unit 40.

As for the imager 41, the imager 42, and the lens 43, only the imager 41 may be disposed, or only the imager 42 and the lens 43 may be disposed.

The light guide member 60 includes a light guide. When the light guide member 60 is contained in the illumination device 20, the illumination device 20 includes a light guide. The light guide member 60 includes an emission section. When the light guide member 60 is contained in the illumination device 20, the illumination device 20 includes an emission section. The emission section is optically connected to the light guide.

Light emitted from the light source 21 is emitted from the lens 22 as illumination light. The beam size of illumination light emitted from the lens 22 is the same as the size of the incident surface of the light guide or larger than the size of the incident surface of the light guide.

Inspection light is used in the light distribution inspection device 10. The inspection light is incident light that is incident on the light guide. The inspection light is part of the illumination light. The beam size of inspection light is smaller than the size of the incident surface of the light guide. The irradiation region is a region irradiated with the inspection light. The irradiation region is narrower than the incident surface of the light guide.

In inspection, the incident position of inspection light relative to the incident surface changes over time. Therefore, a plurality of beams of inspection light are incident on the light guide. Incident position information is acquired for each of the beams of inspection light.

The inspection light incident on the incident surface of the light guide is emitted from an emission surface of the light guide. Since there are a plurality of beams of inspection light, a plurality of beams of emission light are emitted from the emission surface of the light guide. Since the emission surface is located at the emission section of the illumination device 20, the beams of emission light are emitted from the emission section of the illumination device 20. Thus, the light quantity distribution characteristic is acquired for each of the beams of emission light.

As a result, a plurality of pieces of incident position information and a plurality of light quantity distribution characteristics are acquired. The information acquisition unit 40 acquires each of the light quantity distribution characteristics in association with each of pieces of the incident position information.

The light quantity distribution characteristic is acquired based on the emission light emitted from the emission section of the illumination device 20. The emission light is generated from inspection light incident on the light guide of the illumination device 20. Thus, the light quantity distribution characteristic is acquired based on the inspection light incident on the light guide.

The light distribution information calculating unit 50 calculates light distribution information of the illumination device 20. The calculation of the light distribution information is performed based on each piece of the incident position information and each light quantity distribution characteristic.

In the light guide member 60, the number of emission sections is one. The light guide member 60 may have a plurality of emission sections. In this case, the beams of emission light are emitted from the emission sections of the illumination device 20, respectively.

As described later, it is possible to set an endoscope as an inspection target in the light distribution inspection device 10. The endoscope includes an imager and an objective lens. Thus, when the endoscope is inspected, the imager 42 and the lens 43 are not used.

Inspection in Light Distribution Inspection Device

The inspection in the light distribution inspection device 10 will be described. As illustrated in FIG. 3A, when the light source 21 is a point light source, light emitted from the light source 21 is converted into parallel light by the lens 22. Light emitted from the illumination device 20 (hereinafter referred to as “illumination light L_{ILL}”) is incident on the light guide member 60.

The light guide member 60 includes a light guide 61 and a sheath 62. The light guide 61 has an incident surface 63. The light guide member 60 is held by the holding member 30.

As illustrated in FIG. 3B, the light guide 61 includes a plurality of light guide elements 64. The light guide elements 64 are arranged in a grid pattern. The arrangement of the light guide elements may be hexagonal close-packed or random.

For example, it is possible to use optical fibers as the light guide elements 64. In this case, the light guide 61 functions as a fiber bundle.

In FIG. 3A, the DMD 70 is not disposed in order to compare the size of the illumination light L_{ILL} with the size of the incident surface 63. As illustrated in FIG. 3A, the illumination light L_{ILL} has a beam diameter including the incident surface 63 of the light guide 61. Thus, in this state, all of the illumination light L_{ILL} is incident on the incident surface 63.

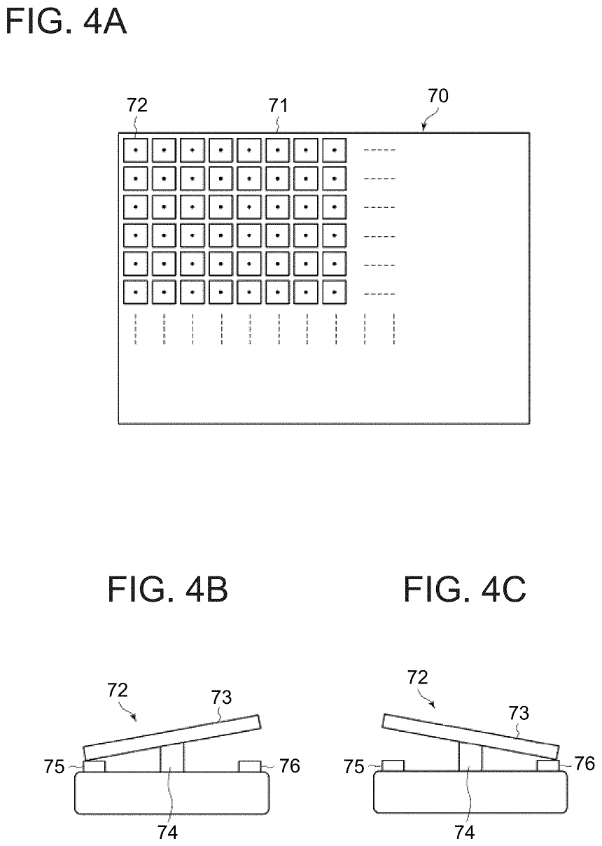

FIG. 4A, FIG. 4B, and FIG. 4C are diagrams illustrating the digital mirror device. FIG. 4A is a diagram illustrating a mirror surface. FIG. 4B is a diagram illustrating a first state. FIG. 4C is a diagram illustrating a second state.

As illustrated in FIG. 4A, the DMD 70 has a mirror array surface 71. On the mirror array surface 71, mirror elements 72 are arranged in a grid pattern.

As illustrated in FIG. 4B and FIG. 4C, each of the mirror elements 72 includes a mirror 73, a hinge 74, an electrode 75, and an electrode 76. The hinge 74 supports the mirror 73 in a tiltable manner. The electrode 75 and the electrode 76 are provided at a position facing the mirror 73.

In the first state, a voltage is applied to the electrode 75 such that attraction is generated between the electrode 75 and the mirror 73. In this case, as illustrated in FIG. 4B, the generated attraction deforms the hinge 74, so that the mirror 73 is tilted. As a result, an end of the mirror 73 comes into contact with the electrode 75.

In the second state, a voltage is applied to the electrode 76 such that attraction is generated between the electrode 76 and the mirror 73. In this case, as illustrated in FIG. 4C, the generated attraction deforms the hinge 74, so that the mirror 73 is tilted. In the second state, the direction of deformation of the hinge 74 and the direction of tilting of the mirror 73 are opposite to those in the first state. Therefore, an end of the mirror 73 comes into contact with the electrode 76.

In the first state, the mirror 73 is inclined approximately +10°. In the second state, the mirror 73 is inclined approximately −10°. In the DMD 70, each of the mirror elements 72 is controlled to be in either one of the first state or the second state.

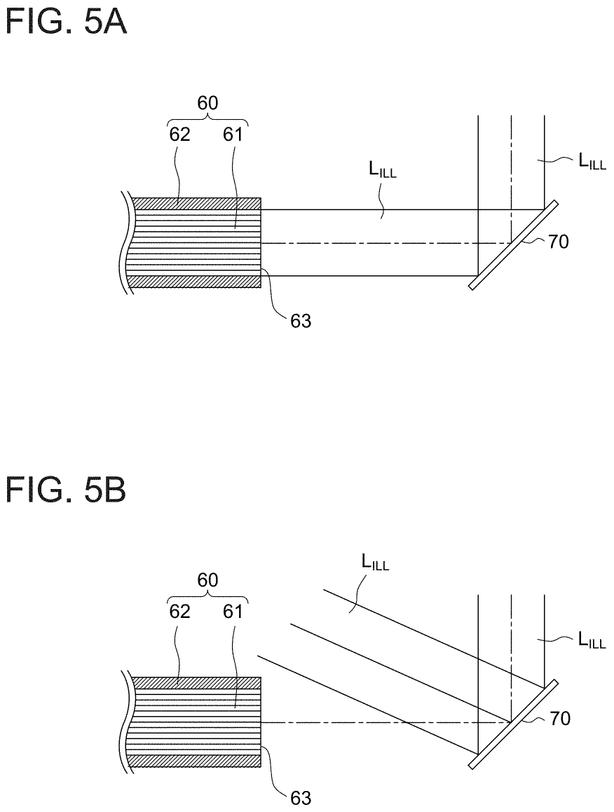

FIG. 5A and FIG. 5B are diagrams illustrating a state in which illumination light is reflected. FIG. 5A is a diagram illustrating illumination light in a first state. FIG. 5B is a diagram illustrating illumination light in a second state.

When all of the mirror elements 72 are in the first state, it is possible to allow all of the illumination light L_{ILL} to be incident on the incident surface 63, as illustrated in FIG. 5A. On the other hand, when all of the mirror elements 72 are in the second state, it is possible to prevent all of the illumination light L_{ILL} from being incident on the incident surface 63, as illustrated in FIG. 5B.

Thus, it is possible to select light to be incident on the incident surface 63 from the illumination light L_{ILL} by bringing some of the mirror elements 72 into the first state and the remaining mirror elements 72 into the second state.

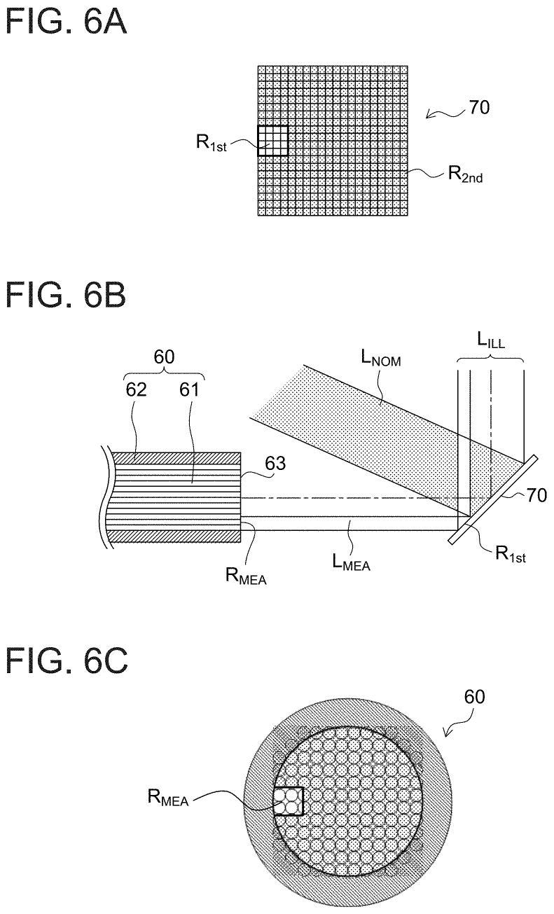

FIG. 6A, FIG. 6B, and FIG. 6C are diagrams illustrating the digital mirror device and inspection light. FIG. 6A is a diagram illustrating the mirror array surface. FIG. 6B is a diagram illustrating a state in which illumination light is reflected by the digital mirror device. FIG. 6C is a diagram illustrating an end surface of the light guide member.

As illustrated in FIG. 6A, in the DMD 70, the mirror array surface is divided into a first reflective region R₁ₛₜ and a second reflective region R_{2nd}. In the first reflective region R₁ₛₜ, each mirror element 72 is in the first state. In the second reflective region R_{2nd}, each mirror element 72 is in the second state. Therefore, the traveling direction of light reflected by the first reflective region R₁ₛₜ is different from the traveling direction of light reflected by the second reflective region R_{2nd}.

As illustrated in FIG. 6B, the light reflected by the first reflective region R₁ₛₜ (hereinafter referred to as “inspection light L_{MEA}”) reaches the incident surface 63. Thus, the inspection light L_{MEA} is incident on the light guide 61. On the other hand, the light reflected by the second reflective region Red (hereinafter referred to as “non-inspection light L_{NOM}”) does not reach the incident surface 63. Thus, the non-inspection light L_{NOM} is not incident on the light guide 61.

As illustrated in FIG. 6B and FIG. 6C, an irradiation region R_{MEA} is irradiated with the inspection light L_{MEA}. The irradiation region R_{MEA} is a region irradiated with the inspection light L_{MEA}, on the incident surface 63. As described above, the illumination light L_{ILL} has a beam diameter including the incident surface 63. The inspection light L_{MEA} is part of the illumination light L_{ILL}. Thus, the irradiation region R_{MEA} is narrower than the incident surface 63. By using the DMD 70, it is possible to change the position of the irradiation region R_{MEA}.

The irradiation region R_{MEA} includes a plurality of light guide elements. Thus, the inspection light L_{MEA} is incident on the light guide elements. In the light distribution inspection device 10, the light guide elements correspond to the mirror elements. However, the light guide elements may correspond to one mirror element.

An LCD may be used instead of the DMD 70. For example, in the arrangement illustrated in FIG. 3A, liquid crystals may be arranged between the lens 22 and the incident surface 63.

The liquid crystal includes a plurality of pixels, and it is possible to select transmission or blocking of light for each pixel. Thus, it is possible to divide the illumination light L_{ILL} into inspection light L_{MEA} and non-inspection light L_{NOM}, in the same manner as in the DMD 70.

When pixels that transmit light are changed, the position of the inspection light L_{MEA} on the incident surface 63 changes. Thus, by changing pixels that transmit light, it is possible to change the position of the irradiation region.

Instead of the DMD 70, an aperture member having a transparent region and an opaque region may be used. When the aperture member is irradiated with illumination light L_{ILL}, the illumination light L_{ILL} is divided into light that passes through the transparent region and light that is blocked by the opaque region. Thus, it is possible to divide the illumination light L_{ILL} into inspection light L_{MEA} and non-inspection light L_{NOM}.

Further, when the aperture member is moved mechanically, the position of the transparent region on the incident surface 63 changes. When the position of the transparent region changes, the position of the inspection light L_{MEA} changes. Thus, by moving the aperture member, it is possible to change the position of the irradiation region.

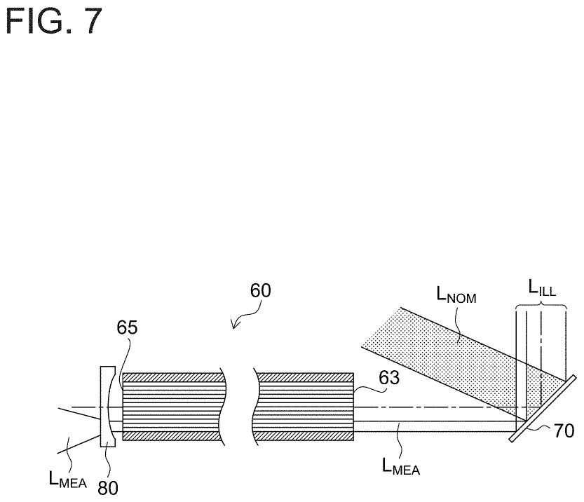

FIG. 7 is a diagram illustrating inspection light emitted from the light guide member. The inspection light L_{MEA} incident on the light guide member 60 is emitted from the light guide member 60. A lens 80 is disposed on the side closer to an emission surface 65 of the light guide member 60. The inspection light L_{MEA} is converted into divergent light by the lens 80. The inspection light L_{MEA} is emitted from the lens 80.

The light guide member that can be inspected is not limited to the light guide member 60. For example, a light guide member with one end surface on the incident side and two or more end surfaces on the emission side can also be inspected.

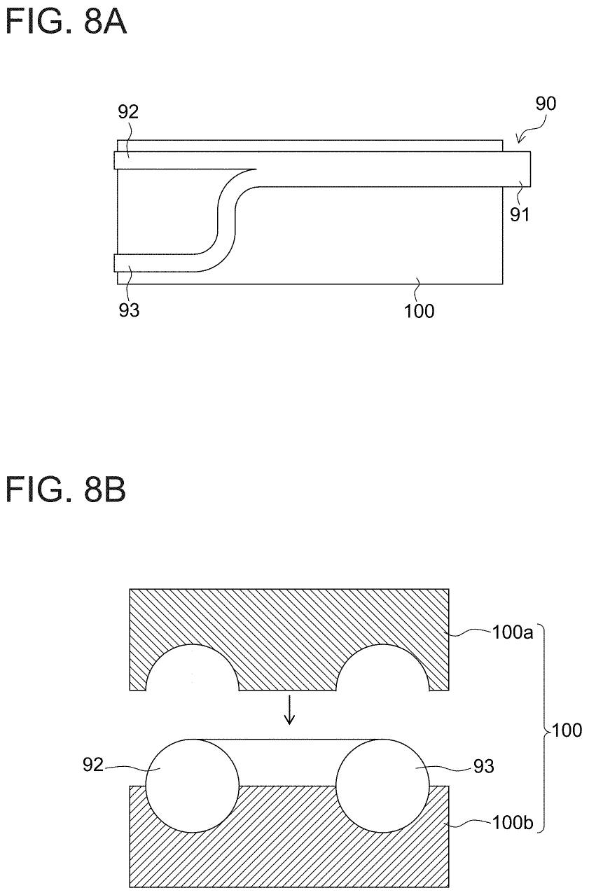

FIG. 8A and FIG. 8B are diagrams illustrating another light guide member. FIG. 8A is a diagram illustrating arrangement of the other light guide member. FIG. 8B is a diagram illustrating a holding member.

As illustrated in FIG. 8A, a light guide member 90 includes a light guide member 91, a light guide member 92, and a light guide member 93. In the light guide member 90, one light guide member is divided into two light guide members in the middle. Therefore, the light guide member 90 has one end surface on the incident side and two end surfaces on the emission side.

The light guide member 90 is held by a holding member 100. As illustrated in FIG. 8B, the holding member 100 includes a first member 100a and a second member 100b. The light guide member 92 and the light guide member 93 are sandwiched between the first member 100a and the second member 100b. The light guide member 90 is held by the holding member 100.

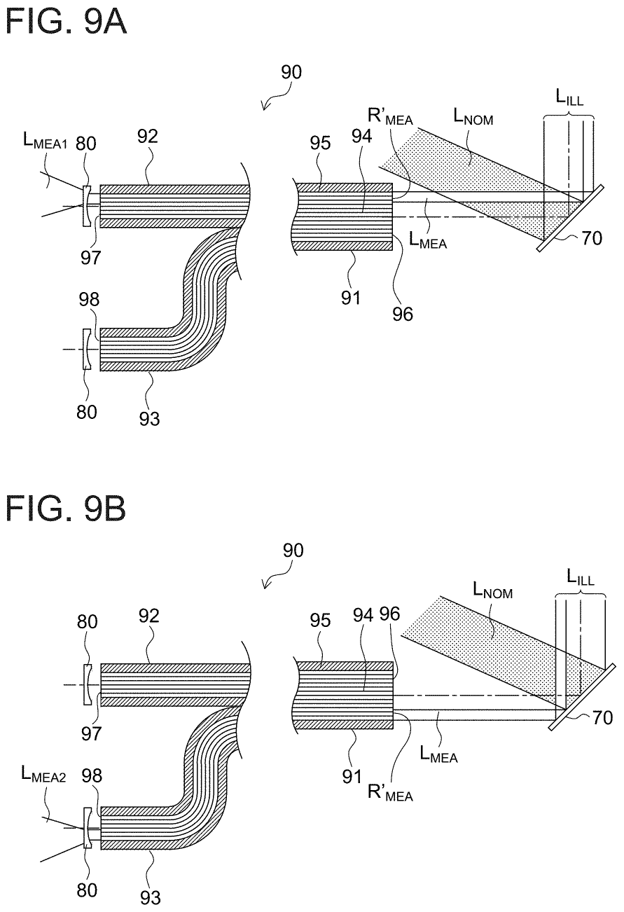

FIG. 9A and FIG. 9B are diagrams illustrating inspection light emitted from the light guide member. FIG. 9A is a diagram illustrating inspection light emitted from one light guide member. FIG. 9B is a diagram illustrating inspection light emitted from the other light guide member.

The light guide member 90 includes a light guide 94 and a sheath 95. The light guide 94 has an incident surface 96. The light guide member 92 has an emission surface 97. The light guide member 93 has an emission surface 98.

An irradiation region R′_{MEA} is irradiated with inspection light L_{MEA}. The irradiation region R′_{MEA} is a region irradiated with the inspection light L_{MEA} on the incident surface 96. Illumination light L_{ILL} has a beam diameter including the incident surface 96. The inspection light L_{MEA} is part of the illumination light L_{ILL}. Thus, the irradiation region R′_{MEA} is narrower than the incident surface 96. It is possible to change the position of the irradiation region R′_{MEA} by using the DMD 70.

The inspection light L_{MEA} is emitted from the light guide member 92 or emitted from the light guide member 93 in accordance with the position of the irradiation region R′_{MEA}. In some cases, the inspection light L_{MEA} is emitted from both of the light guide member 92 and the light guide member 93.

As illustrated in FIG. 9A, when the position of the irradiation region R′_{MEA} is closer to the light guide member 92, the inspection light L_{MEA} incident on the incident surface 96 is emitted from the light guide member 92. The lens 80 is disposed on the side closer to the emission surface 97 of the light guide member 92. The inspection light L_{MEA} emitted from the light guide member 92 is converted into divergent light by the lens 80. Inspection light L_{MEA1} is emitted from the lens 80.

As illustrated in FIG. 9B, when the position of the irradiation region R′_{MEA} is closer to the light guide member 93, the inspection light L_{MEA} incident on the incident surface 96 is emitted from the light guide member 93. The lens 80 is disposed on the side closer to the emission surface 98 of the light guide member 93. The inspection light L_{MEA} emitted from the light guide member 93 is converted into divergent light by the lens 80. Inspection light L_{MEA} is emitted from the lens 80.

In the inspection of the light guide member 60 and the light guide member 90, the light quantity distribution characteristic is acquired based on the inspection light L_{MEA}. In this case, the light quantity distribution characteristic is acquired in association with the incident position information of the inspection light L_{MEA}.

The irradiation region R_{MEA} is a region irradiated with the inspection light L_{MEA} on the incident surface 63. The position of the irradiation region R_{MEA} represents the incident position of the inspection light L_{MEA} on the incident surface 63. Further, the irradiation region R′_{MEA} is a region irradiated with the inspection light L_{MEA} on the incident surface 96. The position of the irradiation region R′_{MEA} represents the incident position of the inspection light L_{MEA} on the incident surface 96.

Thus, it is possible to use information on the position of the irradiation region R_{MEA} and information on the position of the irradiation region R′_{MEA} as the incident position information of the inspection light L_{MEA}.

As illustrated in FIG. 6C, the irradiation region R_{MEA} is a region irradiated with the inspection light L_{MEA}. Since the inspection light L_{MEA} is reflected by the first reflective region R₁ₛₜ, it is possible to use the position of the first reflective region R₁ₛₜ instead of the position of the irradiation region R_{MEA}. Thus, it is possible to use information on the position of the first reflective region R₁ₛₜ as the incident position information of the inspection light L_{MEA}.

The first reflective region R₁ₛₜ represents a region in which the mirror elements are in the first state. Since the mirror elements are arranged in a grid pattern, it is possible to specify the position of each mirror element. By specifying a mirror element included in the first reflective region R₁ₛₜ, it is possible to use the position of the specified mirror element instead of the position of the first reflective region R₁ₛₜ. Thus, it is possible to use information on the position of the mirror elements as the incident position information of the inspection light L_{MEA}.

In the inspection of the light guide member 60, it is preferable that calculation of the light quantity distribution characteristic and the light distribution information be performed for the entire incident surface 63. As described above, the irradiation region R_{MEA} is narrower than the incident surface 63. Thus, calculation of the light quantity distribution characteristic and the light distribution information is performed while the position of the irradiation region R_{MEA} is changed.

In the inspection of the light guide member 90, it is preferable that calculation of the light quantity distribution characteristic and the light distribution information be performed for the entire incident surface 96. As described above, the irradiation region R′_{MEA} is narrower than the incident surface 96. Thus, calculation of the light quantity distribution characteristic and the light distribution information is performed while the position of the irradiation region R′_{MEA} is changed.

In the inspection using the light guide member 60, the inspection light L_{MEA} is emitted from the lens 80. In the inspection using the light guide member 90, the inspection light L_{MEA1} and the inspection light L_{MEA2} are emitted from the lens 80. It is possible to pick up these beams of inspection light using a first method or a second method.

Image Pickup by First Method

It is preferable that the light distribution inspection device of the present embodiment include an imager configured to output a signal to be used to generate an image, and the imager faces a holding member.

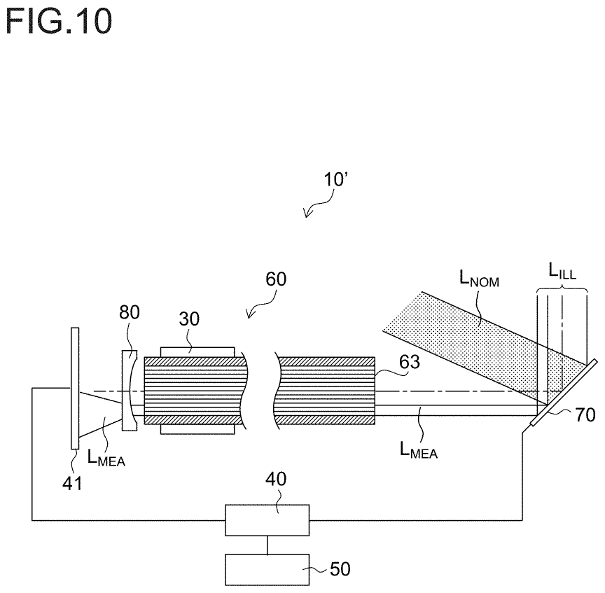

FIG. 10 is a diagram illustrating image pickup by the first method. The same configuration as that in FIG. 2 is denoted by the same number and a description thereof will be omitted. FIG. 10 is a schematic diagram.

As illustrated in FIG. 10, the imager 41 is used in the first method. The imager 42 and the lens 43 are not used.

In a light distribution inspection device 10′, the imager 41 faces the holding member 30. The holding member 30 can hold the light guide member 60 or the light guide member 90.

In FIG. 10, the light guide member 60 is held by the holding member 30. The inspection light L_{MEA} emitted from the light guide member 60 passes through the lens 80 and is directly incident on the imager 41.

Although not illustrated in the diagram, when the light guide member 90 is held by the holding member 30, inspection light L_{MEA1} is emitted from the light guide member 92 and inspection light L_{MEA2} is emitted from the light guide member 93. The inspection light L_{MEA2} and the inspection light L_{MEA1} pass through the lens 80 and are directly incident on the imager 41.

A signal to be used to generate an image is output from the imager 41. An image is generated from the output signal.

The generation of an image is performed in the image generating unit. In the light distribution inspection device 10′, the image generating unit is contained in the information acquisition unit 40. Thus, the imager 41 outputs a signal to be used to generate an image to the information acquisition unit 40.

When the image generating unit is separated from the information acquisition unit 40, the imager 41 outputs a signal to be used to generate an image to the image generating unit. The image generating unit outputs the generated image to the information acquisition unit 40.

Acquisition of the light quantity distribution characteristic is performed in the information acquisition unit 40, and calculation of the light distribution information is performed in the light distribution information calculating unit 50.

The light distribution information calculated in the inspection of the light guide member 60 is used for image pickup in an endoscope (hereinafter referred to as “endoscope A”) equipped with the light guide member 60. In image pickup in the endoscope A, an objective lens A mounted on the endoscope A is used. Thus, in the inspection of the light guide member 60, it is necessary to perform calculation of the light distribution information on the premise that the objective lens A is used.

The light distribution information calculated in the inspection of the light guide member 90 is used for image pickup in an endoscope (hereinafter referred to as “endoscope B”) equipped with the light guide member 90. In image pickup in the endoscope B, an objective lens B mounted on the endoscope B is used. Thus, in the inspection of the light guide member 90, it is necessary to perform calculation of the light distribution information on the premise that the objective lens B is used.

In the first method, the inspection light is picked up by the imager, so that the light distribution information is calculated. However, the inspection light is incident directly on the imager. In other words, in the first method, the light distribution information is calculated without using a lens.

Therefore, in the first method, the light distribution information is calculated in consideration of image pickup in the endoscope equipped with the light guide member. Specifically, the position of the imager and the size of the image pickup surface are determined based on the field of view of the objective lens used with the light guide member.

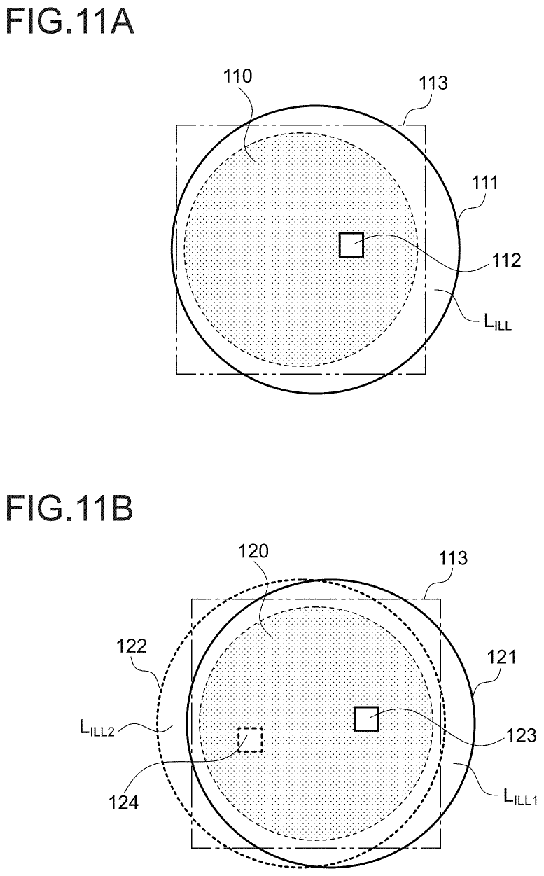

FIG. 11A and FIG. 11B are diagrams illustrating a field of view of an objective lens, an illumination region, and an image pickup region. FIG. 11A is a diagram illustrating a case with a beam of inspection light. FIG. 11B is a diagram illustrating a case with two beams of inspection light.

Case with a Beam of Inspection Light In the light guide member 60, a beam of inspection light is emitted from the light guide member 60. It is possible to use the light guide member 60 in an endoscope. The endoscope includes a fiber bundle, an illumination lens, and an objective lens. The light guide 61 of the light guide member 60 corresponds to the fiber bundle. Further, the lens 80 corresponds to the illumination lens.

In FIG. 11A, an illumination region 111 represents an illumination region of illumination light L_{ILL}. An inspection region 112 represents a region of inspection light L_{MEA}. An image pickup region 113 represents an image pickup region of the imager 41.

In endoscopes, the specifications of the fiber bundle, the specifications of the illumination lens, and the specifications of the objective lens vary with the products. In the inspection of the light guide member 60, the illumination lens of the endoscope A is used for the lens 80.

If necessary, it is possible to use the imager used in the endoscope A as the imager 41. Further, it is possible to use the DMD used in the endoscope A as the DMD 70.

In endoscopes, an observation range is determined by the field of view of the objective lens. If the field of view of the objective lens is not filled with illumination light, it is impossible to observe the inside of the observation range. Further, if the field of view of the objective lens is not included in the image pickup region, it is impossible to observe the inside of the observation range.

In the first method, the objective lens A is not used. For this reason, a field of view 110 of the objective lens A (hereinafter referred to as “field of view 110”) is obtained based on the specifications of the objective lens A. Then, the imager 41 is positioned such that the field of view 110 is included in the image pickup region 113. Further, the size of the image pickup region 113 is determined in consideration of the field of view 110.

The illumination region 111 is formed with the lens 80. The illumination lens of the endoscope A is used as the lens 80. The illumination lens of the endoscope A is designed such that the field of view 110 is filled. Thus, at the position of the imager 41, the illumination region 111 includes the field of view 110, as illustrated in FIG. 11A.

The imager 41 outputs a signal to be used to generate an image to the information acquisition unit 40. The information acquisition unit 40 includes the image generating unit. An image based on the inspection light L_{MEA} (hereinafter referred to as “image IM_{MEA}”) is generated in the image generating unit. Generation of the image IM_{MEA} is performed based on a signal output from the imager 41.

The inspection light L_{MEA} is part of the illumination light L_{ILL}. Therefore, the inspection region 112 is narrower than the field of view 110. Thus, in this state, it is impossible to calculate light distribution information at all the positions in the field of view 110.

For this reason, the light quantity distribution characteristic is acquired while the position of the inspection region 112 is changed. As a result, it is possible to calculate light distribution information at all the positions in the field of view 110. It is possible to change the position of the inspection region 112 by moving the irradiation region.

Case with Two Beams of Inspection Light

It is also possible to use the light guide member 90 in an endoscope. In the light guide member 90, two beams of inspection light are emitted from the light guide member 92 and the light guide member 93. It is possible to use the light guide member 90 in an endoscope. The endoscope includes a fiber bundle, an illumination lens, and an objective lens. The light guide 94 of the light guide member 90 corresponds to the fiber bundle. Further, the lens 80 corresponds to the illumination lens.

In FIG. 11B, an illumination region 121 represents illumination light L_{ILL1}. An illumination region 122 represents an illumination region of illumination light L_{ILL2}. An inspection region 123 represents a region of inspection light L_{MEA1}. An inspection region 124 represents a region of inspection light L_{MEA2}.

As described above, in endoscopes, the specifications of the fiber bundle, the specifications of the illumination lens, and the specifications of the objective lens vary with the products. In the inspection of the light guide member 90, the illumination lens of the endoscope B is used for the lens 80.

If necessary, it is possible to use the imager used in the endoscope B as the imager 41. Further, it is possible to use the DMD used in the endoscope B as the DMD 70.

In the first method, the objective lens B is not used. For this reason, a field of view 120 of the objective lens B (hereinafter referred to as “field of view 120”) is obtained based on the specifications of the objective lens B. Then, the imager 41 is positioned such that the field of view 120 is included in the image pickup region 113. Further, the size of the image pickup region 113 is determined in consideration of the field of view 120.

The illumination region 121 and the illumination region 122 are formed with the lens 80. The illumination lens of the endoscope B is used as the lens 80. The illumination lens of the endoscope B is designed such that the field of view 120 is filled. Thus, at the position of the imager 41, the illumination region 121 and the illumination region 122 include the field of view 120, as illustrated in FIG. 11B.

The imager 41 outputs a signal to be used to generate an image to the information acquisition unit 40. The information acquisition unit 40 includes the image generating unit. An image IM_{MEA} is generated in the image generating unit. The image IM_{MEA} includes an image based on the inspection light L_{MEA1} (hereinafter referred to as “image IM_{MEA1}”) and an image based on the inspection light L_{MEA2} (hereinafter referred to as “image IM_{MEA2}”). Generation of the image IM_{MEA1} and generation of the image IM_{MEA2} are performed based on signals output from the imager 41.

Both of the inspection light L_{MEA1} and the inspection light L_{MEA2} are parts of the illumination light L_{ILL}. Therefore, both of the inspection region 123 and the inspection region 124 are narrower than the field of view 120. Thus, in this state, it is impossible to calculate light distribution information at all the positions in the field of view 120.

For this reason, acquisition of the light quantity distribution characteristic is performed by the information acquisition unit 40 while the position of the inspection region 123 and the position of the inspection region 124 are changed. As a result, it is possible to calculate light distribution information at all the positions in the field of view 120. It is possible to change the position of the inspection region 123 and the position of the inspection region 124 by moving the irradiation region.

In FIG. 11A, the field of view 110 is located inside the image pickup region 113. However, the field of view 110 may be inscribed in the image pickup region 113. Alternatively, the field of view 110 may be circumscribed about the image pickup region 113. These are applicable to the field of view 120.

In the inspection described above, the image pickup region is set to include the field of view of the objective lens. Thus, it is possible to perform inspection with the imager kept fixed. However, an imager having an image pickup region narrower than the field of view may be used.

When an imager having an image pickup region narrower than the field of view is used, a lens may be disposed between the lens 80 and the imager. By doing so, it is possible to collect inspection light emitted from the lens 80. As a result, it is possible to perform inspection with the imager kept fixed even when an imager having an image pickup region narrower than the field of view is used.

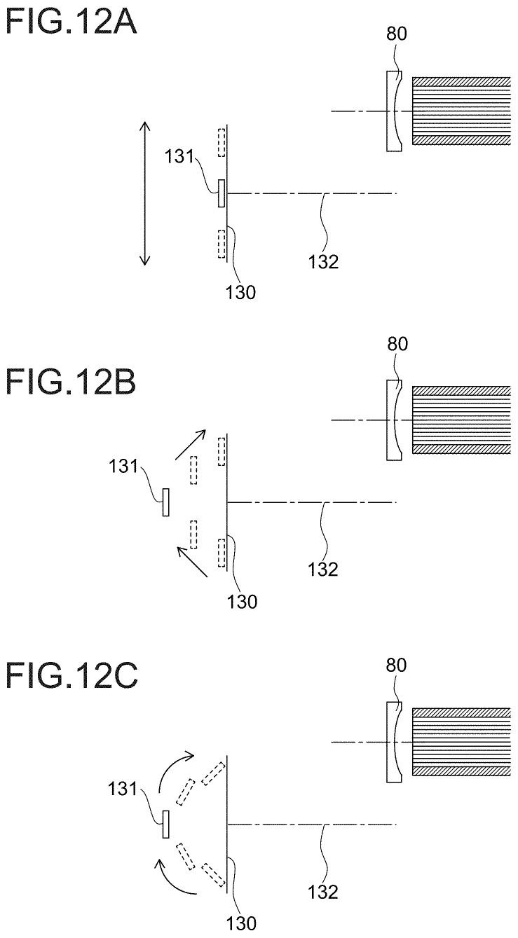

In the inspection, the imager may be moved. FIG. 12A, FIG. 12B, and FIG. 12C are diagrams illustrating movement of an imager. FIG. 12A is a diagram illustrating first movement. FIG. 12B is a diagram illustrating second movement. FIG. 12C is a diagram illustrating third movement.

In the first movement, as illustrated in FIG. 12A, an imager 131 is moved within a field of view 130. The moving direction of the imager 131 is a direction orthogonal to an optical axis 132, as indicated by the arrow.

In the second movement, as illustrated in FIG. 12B, the imager 131 is moved in a direction orthogonal to the optical axis 132 and moved along the optical axis. In the second movement, the direction of the normal to the surface of the imager 131 is not changed.

In the third movement, as illustrated in FIG. 12C, the imager 131 is moved in a direction orthogonal to the optical axis 132 and moved along the optical axis. In the third movement, the direction of the normal to the surface of the imager 131 is changed.

The light-receiving surface of the imager 131 has a largeness that can receive the inspection light emitted from the lens 80. Thus, it is possible to receive the inspection light emitted from the lens 80, irrespective of the position of the imager 131.

With an endoscope, for example, the inner wall of the intestines is observed. In this case, the distance from the objective lens to the inner wall differs between the center of the field of view and the periphery of the field of view. At the periphery of the field of view, the distance to the objective lens is shorter than at the center of the field of view. The distance reached by illumination light is also shorter at the periphery of the field of view and longer at the center of the field of view.

In the second movement method and the third movement method, it is possible to perform inspection in consideration of the difference in distance. Thus, it is possible to perform inspection at high accuracy.

Image Pickup by Second Method

It is preferable that the light distribution inspection device of the present embodiment include an imager configured to output signals to be used to generate images, each of the images is a picked-up image of the inspection light reflected by a reflector, and a holding member and the imager are disposed on one side of the reflector.

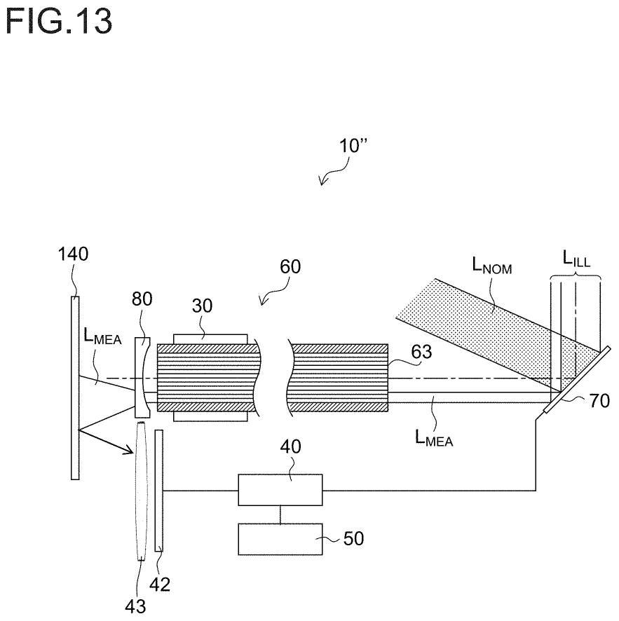

FIG. 13 is a diagram illustrating image pickup by the second method. The same configuration as that in FIG. 2 is denoted by the same number and a description thereof will be omitted. FIG. 13 is a schematic diagram.

As illustrated in FIG. 13, the imager 42 and the lens 43 are used in the second method. The imager 41 is not used. Further, a reflector 140 is used. A specific example of the reflector 140 will be described later.

In a light distribution inspection device 10″, the imager 42 and the lens 43 are proximate to each other. In an actual device, the distance between the imager 42 and the lens 43 is appropriately set so that an optical image can be picked up by the imager 42.

In the light distribution inspection device 10″, the reflector 140 is disposed at a position facing the holding member 30 with the lens 80 interposed therebetween. In this case, inspection light L_{MEA} emitted from the lens 80 is reflected by the reflector 140. Thus, the holding member 30 and the imager 42 are disposed on one side of the reflector 140. The holding member 30 can hold the light guide member 60 or the light guide member 90.

In FIG. 13, the light guide member 60 is held by the holding member 30. The inspection light L_{MEA} emitted from the light guide member 60 is incident on the lens 80. The inspection light L_{MEA} emitted from the lens 80 is reflected by the reflector 140. Part of the reflected inspection light L_{MEA} passes through the lens 43 and is incident on the imager 42.

Although not illustrated in the diagram, when the light guide member 90 is held by the holding member 30, inspection light L_{MEA1} is emitted from the light guide member 92 and inspection light L_{MEA2} is emitted from the light guide member 93. The inspection light L_{MEA2} and the inspection light L_{MEA1} are incident on the lens 80. The inspection light L_{MEA1} and the inspection light L_{MEA2} emitted from the lens 80 are reflected by the reflector 140. Part of the reflected inspection light L_{MEA1} and part of the reflected inspection light L_{MEA2} pass through the lens 43 and are incident on the imager 42.

A signal to be used to generate an image is output from the imager 42. An image is generated from the output signal.

The generation of an image is performed in the image generating unit. In the light distribution inspection device 10″, the image generating unit is contained in the information acquisition unit 40. Thus, the imager 42 outputs a signal to be used to generate an image to the information acquisition unit 40.

When the image generating unit is separated from the information acquisition unit 40, the imager 42 outputs a signal to be used to generate an image to the image generating unit. The image generated by the image generating unit is output to the information acquisition unit 40.

Acquisition of the light quantity distribution characteristic is performed in the information acquisition unit 40, and calculation of the light distribution information is performed in the light distribution information calculating unit 50.

The light distribution information calculated in the inspection of the light guide member 60 is used for image pickup in the endoscope A. Thus, in the inspection of the light guide member 60, it is necessary to perform calculation of the light distribution information on the premise that the objective lens A is used.

The light distribution information calculated in the inspection of the light guide member 90 is used for image pickup in the endoscope B. Thus, in the inspection of the light guide member 90, it is necessary to perform calculation of the light distribution information on the premise that the objective lens B is used.

In the second method, the inspection light is picked up by the imager, so that the light distribution information is calculated. However, the inspection light is incident on the imager through the reflector and the lens. In other words, in the second method, the light distribution information is calculated using a lens.

Even in the second method, the light distribution information is calculated in consideration of image pickup in the endoscope equipped with the light guide member. Specifically, the position of the imager 42, the size of the image pickup surface, the position of the lens 43, the position of the reflector 140, and the size of the reflector 140 are determined such that the same observation range as that of the endoscope can be picked up.

Case with a Beam of Inspection Light

In the light guide member 60, one inspection light is emitted from the light guide member 60. The light guide member 60 is mounted on the endoscope A. The objective lens A is mounted on the endoscope A. Thus, the objective lens A or a lens having the same field of view as that of the objective lens A may be used for the lens 43.

The position of the imager 42, the size of the image pickup surface, the position of the lens 43, the position of the reflector 140, and the size of the reflector 140 are determined such that the same observation range as that of the endoscope A can be picked up.

The imager 42 outputs a signal to be used to generate an image to the information acquisition unit 40. The information acquisition unit 40 includes the image generating unit. An image IM_{MEA} is generated in the image generating unit. The generation of the image IM_{MEA} is performed based on a signal output from the imager 42.

The inspection light L_{MEA} is part of the illumination light L_{ILL}. Therefore, the inspection region is smaller than the field of view. Thus, in this state, it is impossible to calculate light distribution information at all the positions in the field of view.

For this reason, acquisition of the light quantity distribution characteristic is performed by the information acquisition unit 40 while the position of the inspection region is changed. As a result, it is possible to calculate light distribution information at all the positions in the field of view. It is possible to change the position of the inspection region by moving the irradiation region.

Case with Two Beams of Inspection Light

In the light guide member 90, two beams of inspection light are emitted from the light guide member 90. The light guide member 90 is mounted on the endoscope B. The objective lens B is mounted on the endoscope B. Thus, the objective lens B or a lens having the same field of view as that of the objective lens B may be used for the lens 43.

The position of the imager 42, the size of the image pickup surface, the position of the lens 43, the position of the reflector 140, and the size of the reflector 140 are determined such that the same observation range as that of the endoscope B can be picked up.

The imager 42 outputs a signal to be used to generate an image to the information acquisition unit 40. An image is generated. Then, acquisition of the light quantity distribution characteristic is performed in the information acquisition unit 40, and calculation of the light distribution information is performed in the light distribution information calculating unit 50.

The imager 42 outputs a signal to be used to generate an image to the information acquisition unit 40. The information acquisition unit 40 includes the image generating unit. An image IM_{MEA1} and an image IM_{MEA2} are generated in the image generating unit. Generation of the image IM_{MEA1} and generation of the image IM_{MEA2} are performed based on signals output from the imager 42.

Both of the inspection light L_{MEA1} and the inspection light L_{MEA2} are parts of the illumination light L_{ILL}. Therefore, both of two inspection regions are narrower than the field of view. Thus, in this state, it is impossible to calculate light distribution information at all the positions in the field of view.

For this reason, acquisition of the light quantity distribution characteristic is performed by the information acquisition unit 40 while the positions of the two inspection regions are changed. As a result, it is possible to calculate light distribution information at all the positions in the field of view. It is possible to change the positions of the two inspection regions by moving the irradiation region.

As described above, in both of the first method and the second method, inspection light emitted from the lens 80 is incident on the imager. The inspection light is photoelectrically converted by the imager. A signal corresponding to the light quantity distribution characteristic of the inspection light is output from the imager.

The signal output from the imager is input to the image generating unit. In image generation, an image based on the inspection light is generated based on the input signal. The image IM_{MEA}, the image IM_{MEA1}, and the image IM_{MEA2} are images based on inspection light (hereinafter referred to as “image IM”).

In the information acquisition unit 40, the light quantity distribution characteristic is acquired in association with the incident position information. The image IM is used in the acquisition of the light quantity distribution characteristic.

The image IM is associated with the incident position information. The image IM is an image based on inspection light. The position of the inspection light is represented by the position of the irradiation region, the position of the first reflective region, or the position of the mirror element. Thus, the position of the image IM is also represented by these positions. It is possible to associate the image IM with the incident position information.

Further, in the image IM, the light quantity distribution characteristic of the inspection light is imaged. It is possible to acquire the light quantity distribution characteristic from the image IM. Thus, it is possible to acquire the light quantity distribution characteristic in association with the incident position information.

It is possible to acquire the light quantity distribution characteristic from the image IM. Thus, it is possible to acquire the light quantity distribution characteristic in association with the incident position information.

As illustrated in FIG. 6B, the inspection light L_{MEA} is light reflected by the first reflective region R₁ₛₜ of the DMD 70. When the position of the first reflective region R₁ₛₜ changes, the position of the inspection light L_{MEA} on the emission surface of the light guide member 60 also changes. Thus, the image IM_{MEA} generated by the image generating unit also changes. In this way, the position of the first reflective region R₁ₛₜ and the image IM_{MEA} are related.

The DMD 70 is connected to the information acquisition unit 40. The position of the first reflective region R₁ₛₜ is input as incident position information to the information acquisition unit 40. The information acquisition unit 40 has the image IM_{MEA}. Thus, in the information acquisition unit 40, the image IM_{MEA} is associated with the incident position information.

In the image IM_{MEA}, the light quantity distribution characteristic of the inspection light L_{MEA} is imaged. It is possible to acquire the light quantity distribution characteristic from the image IM_{MEA}. Thus, it is possible to acquire the light quantity distribution characteristic in association with the incident position information.

The light quantity distribution characteristic is input together with the incident position information to the light distribution information calculating unit 50. The light quantity distribution characteristic includes the light distribution characteristic of the inspection light L_{MEA} emitted from the light guide 61. Thus, in the information calculating unit 50, it is possible to calculate the light distribution characteristic based on the incident position information and the light quantity distribution characteristic.

The inspection light L_{MEA} reflected by the first reflective region R₁ₛₜ of the DMD 70 has been described above. In the DMD 70, it is possible to select inspection light L_{MEA} to be incident on the incident surface 63 from the illumination light L_{ILL} by bringing some of the mirror elements 72 into the first state and the remaining mirror elements 72 into the second state.

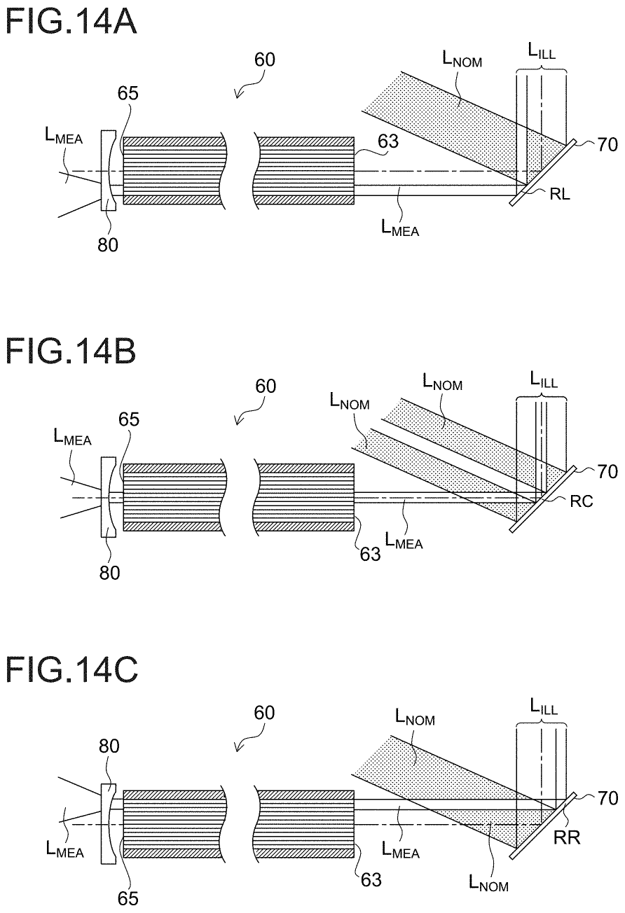

FIG. 14A, FIG. 14B, and FIG. 14C are diagrams illustrating light selected by the digital mirror device. FIG. 14A is a diagram illustrating light in a first selection state. FIG. 14B is a diagram illustrating light in a second selection state. FIG. 14C is a diagram illustrating light in a third selection state.

In the first selection state, a mirror element group RL is in the first state and the remaining mirror element group is in the second state. The mirror element group RL is located at one end of the mirror array surface. Thus, as illustrated in FIG. 14A, inspection light L_{MEA} is emitted from one end of the emission surface 65.

In the second selection state, a mirror element group RC is in the first state and the remaining mirror element group is in the second state. The mirror element group RC is located at the center of the mirror array surface. Thus, as illustrated in FIG. 14B, inspection light L_{MEA} is emitted from the center of the emission surface 65.

In the third selection state, a mirror element group RR is in the first state and the remaining mirror element group is in the second state. The mirror element group RR is located at the other end of the mirror array surface. Thus, as illustrated in FIG. 14C, inspection light L_{MEA} is emitted from the other end of the emission surface 65.

In this way, by using the DMD 70, it is possible to change the position of the inspection light L_{MEA} incident on the incident surface 63. When the position of the inspection light L_{MEA} incident on the incident surface 63 changes, the position of the inspection light L_{MEA} emitted from the emission surface 65 can be changed.

Control of the DMD 70 is performed by the information acquisition unit 40. In addition, in the information acquisition unit 40, it is possible to associate the image IM_{MEA} with the incident position information. Thus, it is possible to perform association of the image with the incident position information and calculation of the light distribution information while the position of the irradiation region is changed.

In inspection, the incident surface 63 is repeatedly irradiated with the inspection light L_{MEA} while the position of the irradiation region relative to the incident surface 63 is changed. The irradiation of the inspection light L_{MEA} is performed until the entire incident surface 63 is irradiated with the inspection light L_{MEA}.

As described above, it is possible to use the light guide member 60 and the light guide member 90 for fiber bundles of an endoscope. Thus, in the light distribution inspection device of the present embodiment, it is possible to perform inspection using an endoscope.

In inspection, it is preferable that the mirror elements be brought into the first state one by one. However, a plurality of mirror elements may be brought into the first state simultaneously. For example, the mirror elements may be brought into the first state one row at a time. By doing so, it is possible to efficiently perform inspection.

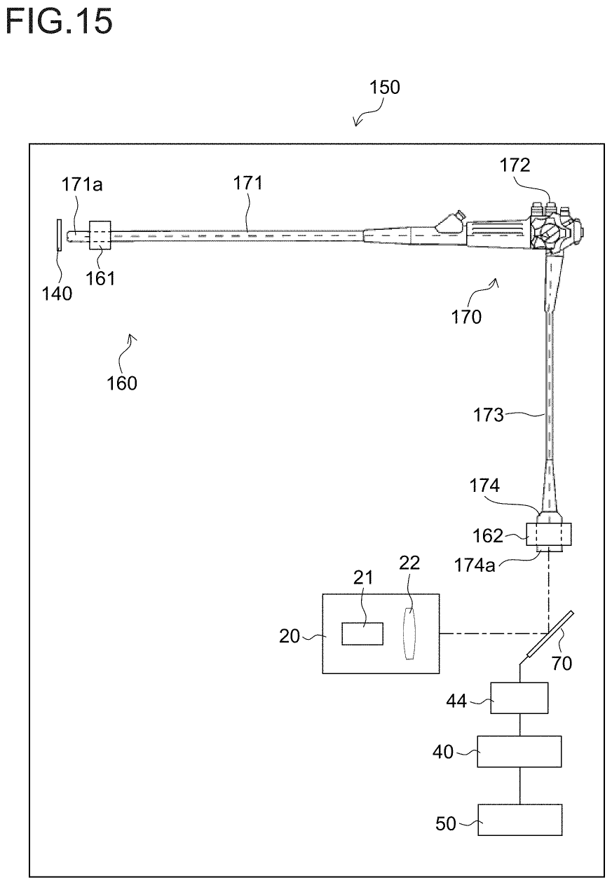

FIG. 15 is a diagram illustrating a light distribution inspection device. The same configuration as that in FIG. 2 is denoted by the same number and a description thereof will be omitted.

A light distribution inspection device 150 includes the illumination device 20, a holding member 160, the information acquisition unit 40, and the light distribution information calculating unit 50. An inspection target is an endoscope 170.

The endoscope 170 includes an insertion section 171, an operating section 172, a cable 173, and a connection section 174. The insertion section 171 has a distal end 171a. A fiber bundle, an illumination lens, an objective lens, and an imager are disposed in the distal end 171a.

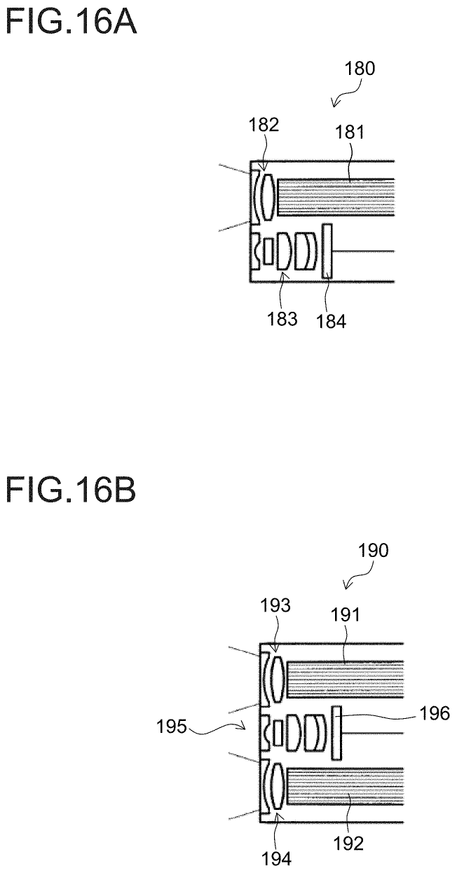

The number of fiber bundles and the number of objective lenses vary depending on the purpose of use of the endoscope. FIG. 16A and FIG. 16B are diagrams illustrating a distal end of an endoscope. FIG. 16A is a diagram illustrating a first example of the distal end. FIG. 16B is a diagram illustrating a second example of the distal end.

As illustrated in FIG. 16A, a distal end 180 in the first example includes a fiber bundle 181, an illumination lens 182, an objective lens 183, and an imager 184. A first distal end 180 has one emission surface. Thus, in the first distal end 180 in the first example, inspection light is emitted from one direction.

As illustrated in FIG. 16B, a distal end 190 in the second example includes a fiber bundle 191, a fiber bundle 192, an illumination lens 193, an illumination lens 194, an objective lens 195, and an imager 196. The distal end 190 in the second example has two emission surfaces. Thus, in the distal end 190 in the second example, inspection light is emitted from two directions.

Returning to FIG. 15, a description will be given. The fiber bundle and a signal line of the imager extend from the distal end 171a to the connection section 174. An incident surface of the fiber bundle is located on a connection surface 174a of the connection section 174. The signal line of the imager is connected to the information acquisition unit 40 through a connector (not illustrated).

The holding member 160 includes a holding member 161 and a holding member 162. The insertion section 171 is held by the holding member 161. The connection section 174 is held by the holding member 162. The fiber bundle is disposed in the inside of the insertion section 171 and the inside of the connection section 174. Thus, the fiber bundle is held by the holding member 161 and the holding member 162.

In the endoscope 170, the imager and the fiber bundle are located on one side. Thus, the reflector 140 is used in inspection. In this way, in the light distribution inspection device 150, inspection is performed using the second method. The reflector will be described.

It is preferable that the reflector have a region in which the inspection light can be picked up by the imager.

In the second method, the inspection light emitted from the light guide member is incident on the reflector and thereafter reflected by the reflector. The reflector has a region in which the inspection light can be picked up by the imager, whereby the inspection light emitted from the light guide member is reflected toward the imager. Thus, it is possible to perform inspection based on the inspection light emitted from the light guide member.

Whether the inspection light reflected by the reflector is picked up by the imager is determined by either one of the incident position or the reflection angle, or both.

When the inspection light reflected by the reflector is picked up, one of the following (A1), (A2), and (A3) occurs.

(A1) All beams of the inspection light is picked up by the imager.
(A2) Some of the beams of inspection light are picked up by the imager.
(A3) None of the beams of inspection light is picked up by the imager.

Reflector in First Example

It is preferable that the reflector have a recess, an inner surface of the recess have a reflective region formed of a plurality of reflective surfaces, and the reflective region have a largeness that allows inspection light emitted from the light guide member to be incident on the reflective region.

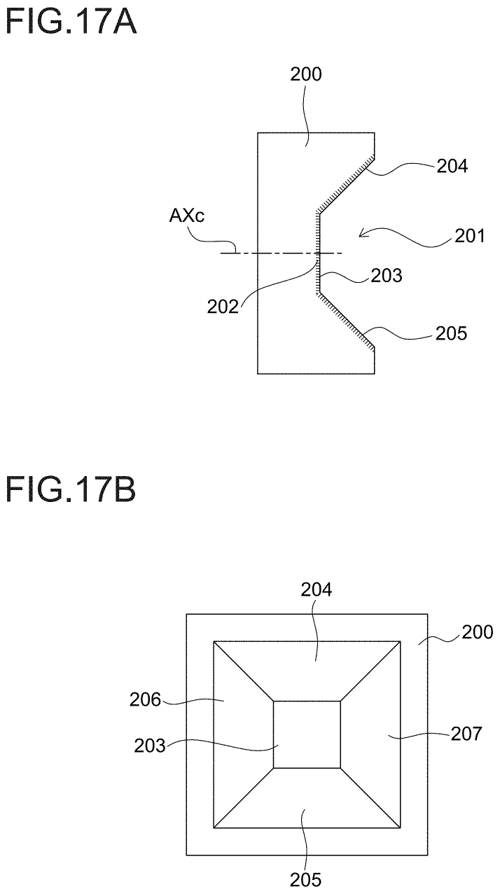

FIG. 17A and FIG. 17B are diagrams illustrating a reflector in a first example. FIG. 17A is a side view. FIG. 17B is a top view.

As illustrated in FIG. 17A, a reflector 200 has a recess 201. The inner surface of the recess 201 has a reflective region 202. The reflective region 202 is formed of a plurality of reflective surfaces. As illustrated in FIG. 17B, the reflective region 202 is formed of a reflective surface 203, a reflective surface 204, a reflective surface 205, a reflective surface 206, and a reflective surface 207.

Although not illustrated in the diagram, when the reflector 200 is in use, the recess 201 faces the holding member. The light guide member is held in the holding member. The reflective region 202 is irradiated with inspection light emitted from the light guide member. The reflective region 202 has a largeness that allows the inspection light emitted from the light guide member to be incident on the reflective region.

It is preferable that each of the reflective surfaces be planar.

In the reflector 200, the reflective surface 203, the reflective surface 204, the reflective surface 205, the reflective surface 206, and the reflective surface 207 are planar. By forming the reflective region 202 with a plurality of flat surfaces, it is possible to produce the reflector 200 easily at high accuracy. Thus, it is possible to perform inspection at high accuracy.

It is preferable that the direction of the normal to the reflective surface differ among the reflective surfaces.

In the reflector 200, the direction of the normal to the reflective surface 203 is parallel to a center axis AXc of the reflector 200. The reflective surface 204, the reflective surface 205, the reflective surface 206, and the reflective surface 207 are inclined relative to the reflective surface 203. The tilt angle relative to the reflective surface 203 is 45°. However, the tilt angle is not limited to 45°.

The reflective surface 204 faces the reflective surface 205. Thus, the direction of the normal to the reflective surface 204 differs from the direction of the normal to the reflective surface 205. The reflective surface 206 faces the reflective surface 207. Thus, the direction of the normal to the reflective surface 206 differs from the direction of the normal to the reflective surface 207.

The line connecting the center of the reflective surface 204 and the center of the reflective surface 205 is orthogonal to the line connecting the center of the reflective surface 206 and the center of the reflective surface 207. Thus, the direction of the normal differs among the reflective surface 204, the reflective surface 205, the reflective surface 206, and the reflective surface 207.

It is preferable that each of the reflective surfaces be planar, and the normal to the reflective surface be inclined in each of the reflective surfaces.

The normal to the reflective surface 204, the normal to the reflective surface 205, the normal to the reflective surface 206, and the normal to the reflective surface 207 are inclined relative to the center axis AXc.

The normal to the reflective surface 203 is parallel to the center axis AXc. Therefore, on the reflective surface 203, the distance from the holding member to the reflective surface does not vary at each point on the reflective surface. The normal to the reflective surface 204 is inclined relative to the center axis AXc. Therefore, on the reflective surface 204, the distance from the holding member to the reflective surface varies at each point on the reflective surface.

In the normal to the reflective surface 205, the normal to the reflective surface 206, and the reflective surface 207, the distances from the holding member to the reflective surfaces also vary at each point on the reflective surfaces.

As described above, with an endoscope, for example, the inner wall of the intestines is observed. In this case, the distance from the objective lens to the inner wall differs between the center of the field of view and the periphery of the field of view. At the periphery of the field of view, the distance is shorter than at the center of the field of view. The distance reached by illumination light is also shorter at the periphery of the field of view and longer at the center of the field of view.

With the reflector 200, it is possible to perform inspection in consideration of the difference in distance. Thus, it is possible to perform inspection at high accuracy.

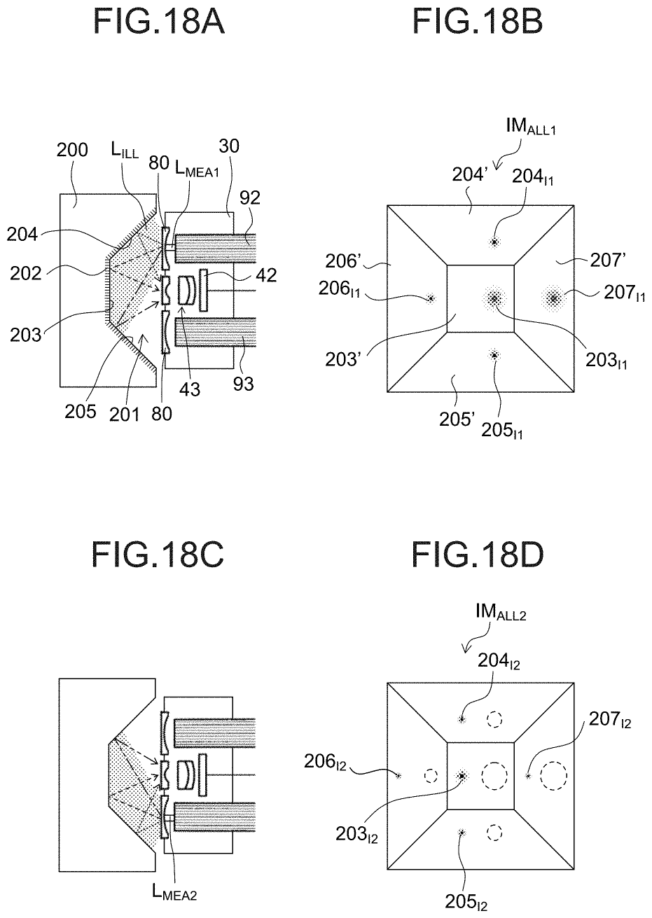

FIG. 18A, FIG. 18B, FIG. 18C, and FIG. 18D are diagrams illustrating the reflector in the first example and an image with inspection light. FIG. 18A is a diagram illustrating inspection light L_{MEA1}. FIG. 18B is a diagram illustrating an image IM_{ALL1}. FIG. 18C is a diagram illustrating inspection light L_{MEA2}. FIG. 18D is a diagram illustrating an image IM_{ALL2}.

The inspection light L_{MEA1} will be described. As illustrated in FIG. 18A, the recess 201 faces the holding member 30. The light guide member 92 and the light guide member 93 are held by the holding member 30. The reflective region 202 is irradiated with the inspection light L_{MEA1} emitted from the light guide member 92.

In FIG. 18A, the inspection light L_{MEA1} is emitted from the center of the light guide member 92. The inspection light L_{MEA1} emitted from the light guide member 92 is reflected by the reflective region 202. The reflective region 202 has a largeness that allows the illumination light L_{ILL} to be reflected. Thus, even when the inspection light L_{MEA1} is emitted from the periphery of the light guide member 92, the reflective region 202 is irradiated with the inspection light L_{MEA1}.

The inspection light L_{MEA1} reflected by the reflective region 202 passes through the lens 43 and is incident on an imager 42. The reflective region 202 is located in the field of view of the lens 43. The reflective region 202 has five reflective surfaces. Thus, as illustrated in FIG. 18B, the entire image IM_{ALL1} includes five regions.

A region 203′ is a region corresponding to the reflective surface 203. A region 204′ is a region corresponding to the reflective surface 204. A region 205′ is a region corresponding to the reflective surface 205. A region 206′ is a region corresponding to the reflective surface 206. A region 207′ is a region corresponding to the reflective surface 207.

When the five reflective surfaces are irradiated with the inspection light L_{MEA1}, and the inspection light L_{MEA1} reflected by each reflective surface is incident on the imager 44, an image with the inspection light L_{MEA1} is included in each of the five regions.

An image 203_{I1} is an image with the inspection light L_{MEA1} reflected by the reflective surface 203. An image 204_{I1} is an image with the inspection light L_{MEA1} reflected by the reflective surface 204. An image 205_{I1} is an image with the inspection light L_{MEA1} reflected by the reflective surface 205. An image 206_{I1} is an image with the inspection light L_{MEA1} reflected by the reflective surface 206. An image 207_{I1} is an image with the inspection light L_{MEA1} reflected by the reflective surface 207.

In FIG. 18B, the image 203_{I1}, the image 204_{I1}, the image 205_{I1}, the image 206_{I1}, and the image 207_{I1} form the image IM_{ALL1}.

In each image, the shape and the light quantity distribution characteristic of the inspection light L_{MEA1} are imaged. The shape and the light quantity distribution characteristic are determined by the shape and the light quantity distribution characteristic of the inspection light L_{MEA1} incident on the imager 42.

In the image IM_{ALL1} illustrated in FIG. 18B, the image 203_{I1} is larger than the image 204_{I1}. This indicates that the inspection light L_{MEA1} reflected by the reflective surface 203 is larger than the inspection light L_{MEA1} reflected by the reflective surface 204.

The inspection light L_{MEA2} will be described. In FIG. 18C, the inspection light L_{MEA2} is emitted from the center of the light guide member 93. The inspection light L_{MEA2} emitted from the light guide member 93 is reflected by the reflective region 202. The reflective region 202 has a largeness that allows the illumination light L_{ILL} to be reflected. Thus, even when the inspection light L_{MEA2} is emitted from the periphery of the light guide member 93, the reflective region 202 is irradiated with the inspection light L_{MEA2}.

The inspection light L_{MEA2} reflected by the reflective region 202 passes through the lens 43 and is incident on the imager 42. The reflective region 202 is located in the field of view of the lens 43. As described above, the reflective region 202 has the five reflective surfaces. Thus, as illustrated in FIG. 18D, the entire image IM_{ALL2} includes five regions.

In FIG. 18D, an image 203_{I2}, an image 204_{I2}, an image 205_{I2}, an image 206_{I2}, and an image 207_{I2} form the image IM_{ALL2}.

In FIG. 18D, the image IM_{ALL1} is indicated by a broken line for reference. The position from which the inspection light L_{MEA2} is emitted is different from the position from which the inspection light L_{MEA1} is emitted. Therefore, even when the beams of inspection light are reflected by the same reflective surface, the position of the image with the inspection light L_{MEA2} is different from the position of the image with the inspection light L_{MEA1}. In some cases, the size of the image with the inspection light L_{MEA2} is also different from the size of the image with the inspection light L_{MEA1}.

Even when a reflective surface is irradiated with the inspection light L_{MEA1}, not all of the inspection light L_{MEA1} reflected by the reflective surface is incident on the imager 42 in some cases. In this case, the region corresponding to the reflective surface does not include the image with the inspection light L_{MEA1}.

Further, even when a reflective surface is irradiated with the inspection light L_{MEA2}, not all of the inspection light L_{MEA2} reflected by the reflective surface is incident on the imager 42 in some cases. In this case, the region corresponding to the reflective surface does not include the image with the inspection light L_{MEA2}.

Therefore, in some cases, the five regions are divided into regions including the image with the inspection light and regions not including the image with the inspection light.

Reflector in Second Example

It is preferable that the reflector have a protrusion and the protrusion is projecting toward the center of a recess.

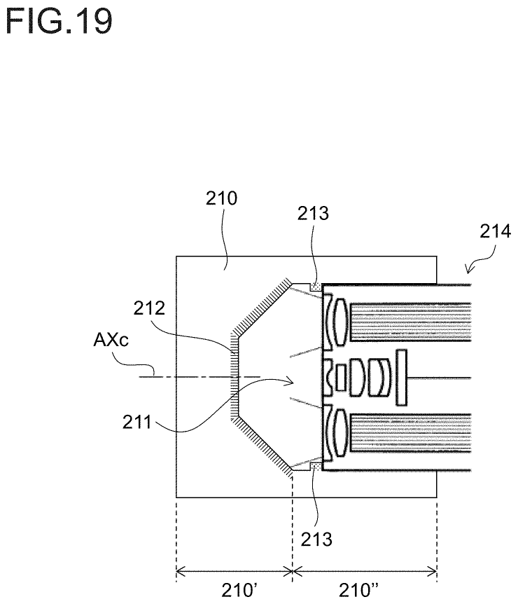

FIG. 19 is a diagram illustrating a reflector in a second example. A reflector 210 has a reflective portion 210′ and a cylindrical portion 210″. In the reflector 210, the reflective portion 210′ and the cylindrical portion 210″ are integrated. However, the reflective portion 210′ and the cylindrical portion 210″ may be separate.

The reflective portion 210′ and the cylindrical portion 210″ form a recess 211. A reflective region 212 is formed in the reflective portion 210′. The cylindrical portion 210″ has protrusions 213. The protrusions 213 are formed on the inner peripheral surface of the cylindrical portion 210″. The protrusions 213 are projecting toward the center axis AXc of the reflector 210.

The center axis AXc is located at the center of the recess 211. Thus, the protrusions 213 are projecting toward the center of the recess 211.

As described above, it is possible to use an endoscope in the inspection with the light distribution inspection device. In the endoscope, inspection light is emitted from a distal end 214. The outer surface of the distal end 214 is a cylindrical surface.

The inner peripheral surface of the cylindrical portion 210″ is a cylindrical surface. The outer diameter of the cylindrical surface is substantially the same as the outer diameter of the distal end 214. Thus, the distal end 214 is inserted into the cylindrical portion 210″, whereby the reflector 210 is attached to the distal end 214.

The cylindrical portion 210″ has the protrusions 213. Thus, by bringing the distal end 214 into contact with the protrusions 213, it is possible to position the reflector 210 relative to the distal end 214.

Reflector in Third Example

It is preferable that the reflector have a connection portion and the holding member is connected to the reflector through the connection portion.

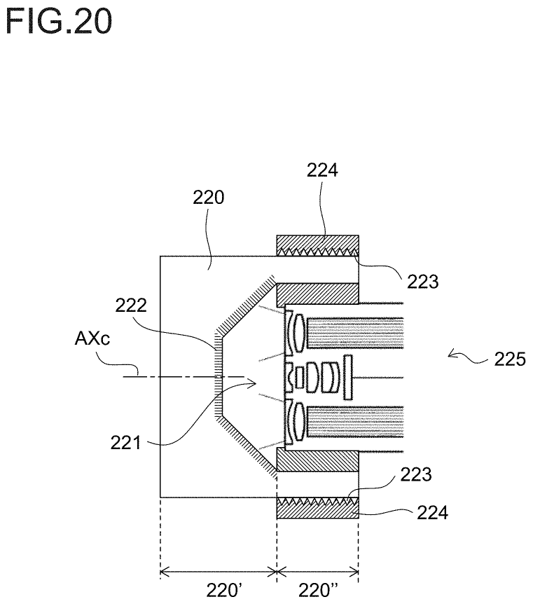

FIG. 20 is a diagram illustrating a reflector in a third example. A reflector 220 has a reflective portion 220′ and a cylindrical portion 220″. In the reflector 220, the reflective portion 220′ and the cylindrical portion 220″ are integrated. However, the reflective portion 220′ and the cylindrical portion 220″ may be separate.

The reflective portion 220′ and the cylindrical portion 220″ form a recess 221. A reflective region 222 is formed in the reflective portion 220′. The cylindrical portion 220″ has a connection portion 223. The connection portion 223 is formed on the outer peripheral surface of the cylindrical portion 220″.

A holding member 224 is connected to the reflector 220 through the connection portion 223. The holding member 224 also has a connection portion. It is possible to connect the reflector 220 and the holding member 224, for example, by a screw. It is possible to hold a distal end 225 of the endoscope in the holding member 224.

As described above, it is possible to use an endoscope in the inspection with the light distribution inspection device. In the endoscope, inspection light is emitted from the distal end 225. The outer surface of the distal end 225 is a cylindrical surface.

The inner peripheral surface of the holding member 224 is a cylindrical surface. The outer diameter of the cylindrical surface is substantially the same as the outer diameter of the distal end 225. Thus, the distal end 225 is inserted into the holding member 224, whereby the distal end 225 is attached to the holding member 224.

The holding member 224 is connected to the reflector 220 through the connection portion 223. Thus, the reflector 220 is attached to the distal end 225. Thus, it is possible to position the reflector 220 relative to the distal end 225.

When the reflector 220 and the holding member 224 are connected by a screw, it is possible to adjust the distance between the reflective region 222 and the holding member 224 by rotating the holding member 224. It is possible to easily perform positioning of the reflector 220 relative to the distal end 225.

The outer diameter of the distal end varies according to the specifications of the endoscope. In the reflector in the second example, the reflector must be prepared in accordance with the outer diameter of the distal end. In the reflector in the third example, the holding member may be prepared in accordance with the outer diameter of the distal end. Therefore, it is possible to use the same reflector even when the outer diameter of the distal end varies.

It is preferable that the reflector be a single reflective surface.

When a single reflective surface is used, the position, the size, and the shape of the reflective surface may be set such that the illumination region and the image pickup region include the field of view. By doing so, it is possible to perform inspection at high accuracy although the structure of the reflector is simple.

Reflector in Fourth Example

It is preferable that the reflector have a mechanism configured to change the direction of the normal to the reflective surface.

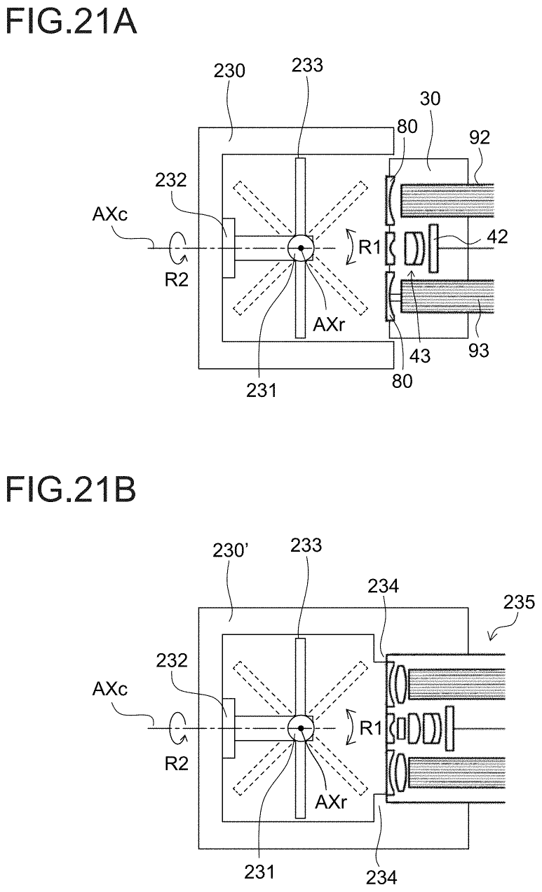

FIG. 21A and FIG. 21B are diagrams illustrating reflectors in a fourth example. FIG. 21A is a diagram illustrating a case where the inspection target is a light guide member. FIG. 21B is a diagram illustrating a case where the inspection target is an endoscope.

As illustrated in FIG. 21A, a reflector 230 includes a first rotating mechanism 231 and a second rotating mechanism 232. A reflective plate 233 is held by the first rotating mechanism 231. The reflective plate 233 has a reflective surface.

As indicated by an arrow R1, the reflective plate 233 moves around a rotation axis AXr. The first rotating mechanism 231 is held by the second rotating mechanism 232. As indicated by an arrow R2, the first rotating mechanism 231 moves around the center axis AXc. As a result, it is possible to change the direction of the normal to the reflective surface.

With the reflector 230, inspection is performed using a light guide member. The reflective plate 233 faces the holding member 30. The light guide member 92 and the light guide member 93 are held by the holding member 30. The reflective plate 233 is irradiated with inspection light emitted from the light guide member 92 and inspection light emitted from the light guide member 93.

The inspection light reflected by the reflective surface passes through the lens 43 and is incident on the imager 42. A signal corresponding to the light quantity distribution characteristic of the inspection light is output from the imager 42. It is possible to acquire an image with the inspection light based on the output signal.

Some light guide members have a narrow light distribution of illumination. With a light guide member with a narrow light distribution of illumination, it is impossible to perform sufficient inspection if the direction of the normal to the reflective surface is limited. With the reflector 230, it is possible to continuously change the direction of the normal to the reflective surface using the first rotating mechanism 231 and the second rotating mechanism 232. Thus, it is possible to perform sufficient inspection even with a light guide member with a narrow light distribution of illumination.

It is also possible to use the reflector 230 for inspection of a light guide member with a wide light distribution of illumination. By using the reflector 230, it is possible to increase the kinds of light guide members to be inspected.

As illustrated in FIG. 21B, a reflector 230′ includes a first rotating mechanism 231 and a second rotating mechanism 232. A reflective plate 233 is held by the first rotating mechanism 231.

With the reflector 230′, inspection is performed using an endoscope. In the reflector 230′, protrusions 234 are formed. By bringing a distal end 235 into contact with the protrusions 234, it is possible to position the reflector 230′ relative to the distal end 235.

Some endoscopes have a narrow light distribution of illumination. With an endoscope with a narrow light distribution of illumination, it is impossible to perform sufficient inspection if the direction of the normal to the reflective surface is limited. With the reflector 230′, it is possible to continuously change the direction of the normal to the reflective surface using the first rotating mechanism 231 and the second rotating mechanism 232. Thus, it is possible to perform sufficient inspection even with an endoscope with a narrow light distribution of illumination.

It is also possible to use the reflector 230′ for inspection of an endoscope with a wide light distribution of illumination. By using the reflector 230′, it is possible to increase the kinds of endoscopes to be inspected.

With the reflector 230 and the reflector 230′, inspection is performed while the direction of the normal to the reflective surface is changed. A not-illustrated drive force supply source is provided for driving the first rotating mechanism 231 and the second rotating mechanism 232. Further, a drive timing communication cable is prepared when inspection is performed automatically.

Reflector in Fifth Example

It is preferable that a first reflector and a second reflector be provided, the first reflector have a first reflective surface, the second reflector have a second reflective surface, the first reflective surface and the second reflective surface each have a region in which the inspection light can be picked up by the imager, and the direction of the normal to the second reflective surface is different from the direction of the normal to the first reflective surface.

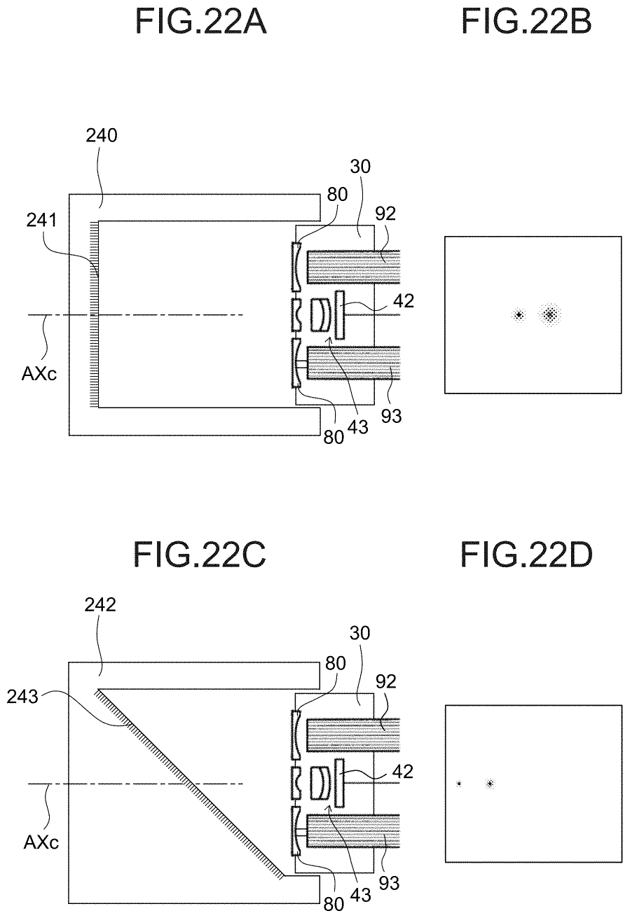

FIG. 22A, FIG. 22B, FIG. 22C, and FIG. 22D are diagrams illustrating reflectors in a fifth example. FIG. 22A is a diagram illustrating a first reflector. FIG. 22B is a diagram illustrating an image obtained with the first reflector. FIG. 22C is a diagram illustrating a second reflector. FIG. 22D is a diagram illustrating an image obtained with the second reflector.

As illustrated in FIG. 22A, a first reflector 240 has a first reflective surface 241. The first reflective surface 241 has a region in which the inspection light can be picked up by the imager 42. The normal to the first reflective surface 241 is parallel to the center axis AXc.

The direction of the normal to the first reflective surface 241 is the same as the direction of the normal to the reflective surface 203 illustrated in FIG. 17B. Thus, as illustrated in FIG. 22B, an image obtained with the first reflector 240 is similar to the image in the region 203′ illustrated in FIG. 18B and FIG. 18D.

As illustrated in FIG. 22C, a second reflector 242 has a second reflective surface 243. The second reflective surface 243 has a region in which the inspection light can be picked up by the imager 42. The normal to the second reflective surface 243 is inclined relative to the center axis AXc. Thus, the direction of the normal to the second reflective surface 243 is different from the direction of the normal to the first reflective surface 241.

In the second reflector 242, the tilt angle of the second reflective surface 243 relative to the center axis AXc is 45°. However, the tilt angle is not limited to 45°.

The direction of the normal to the second reflective surface 243 is the same as the direction of the normal to the reflective surface 205 illustrated in FIG. 17B. Thus, as illustrated in FIG. 22D, an image obtained with the second reflector 242 is similar to the image in the region 205′ illustrated in FIG. 18B and FIG. 18D.

By rotating the second reflector 242 around the center axis AXc by 90° from the state illustrated in FIG. 22B, it is possible to make the direction of the normal to the second reflective surface 243 equal to the direction of the normal to the reflective surface 206 or the direction of the normal to the reflective surface 207.

To make the direction of the normal to the second reflective surface 243 equal to the direction of the normal to the reflective surface 204, the second reflector 242 may be rotated around the center axis AXc by 180° from the state illustrated in FIG. 22B.

By rotating the second reflector 242 around the center axis AXc, it is possible to continuously change the direction of the normal to the second reflective surface 243.

The generation of images based on the inspection light has been described above. Images based on the inspection light are used to calculate light distribution information. Analysis is performed using the calculated light distribution information. The analysis of light distribution information will be described.

Analysis of Light Distribution Information

In the light distribution inspection device of the present embodiment, it is preferable that the light distribution information calculating unit analyze the light distribution information to calculate light quantity and luminous intensity distribution.

In the image generating unit, an image based on inspection light is generated. The image is stored in the information acquisition unit. In the light distribution information calculating unit, light distribution information is calculated using the image based on inspection light. The image based on inspection light is formed of a plurality of pixels. By analyzing the pixels, it is possible to calculate light quantity and luminous intensity distribution. It is possible to represent the luminous intensity distribution by a principal axis and an aspect ratio.

Examples of analysis results are listed in Table 1. The analysis results are obtained when the light guide member 90 illustrated in FIG. 9A and the reflector 200 illustrated in FIG. 17B are used.

The azimuth angle is the angle with respect to the direction of the normal to the reflective surface, and the inclination angle is the angle between the center axis AXc and the normal. The first emission surface is the emission surface 97, and the second emission surface is the emission surface 98. The numerical values in Table 1 represent light quantity. The total represents the total light quantity.

TABLE 1

| | FIRST | SECOND | |
|---|---|---|---|
| AZIMUTH ANGLE/ | EMISSION | EMISSION | REFLECTOR |
| INCLINATION ANGLE | SURFACE | SURFACE | 200 |
| —/0° | 5 | 15 | REFLECTIVE |
| | | | SURFACE 203 |
| 0°/45° | 1 | 10 | REFLECTIVE |
| | | | SURFACE 204 |
| 90°/45° | 2 | 10 | REFLECTIVE |
| | | | SURFACE 205 |
| 180°/45° | 4 | 5 | REFLECTIVE |
| | | | SURFACE 206 |
| 270°/45° | 2 | 10 | REFLECTIVE |
| | | | SURFACE 207 |
| SUM | 14 | 50 | |

In the first emission surface, the total value is 14, and the total light quantity is 14. Further, the light quantity at the reflective surface 203 is the maximum. When the direction of the normal to the surface with the maximum light quantity is the principal axis, the principal axis in the first emission surface is 0′.

Further, the direction connecting the reflective surface 204 and the reflective surface 206 is defined as an X direction, and the direction connecting the reflective surface 205 and the reflective surface 207 is defined as a Y direction. In this case, the total value of the light quantity in the X direction is 5, and the total value of the light quantity in the Y direction is 4. Thus, it can be said that there is a slight bias in the X direction in the light quantity distribution.

In the second emission surface, the total value is 50, and the total light quantity is 50. Further, the light quantity at the reflective surface 203 is the maximum, and the principal axis in the second emission surface is 0°.

Further, the total of the light quantity in the X direction is 15, and the total of the light quantity in the Y direction is 20. Thus, it can be said that there is a slight bias in the Y direction in the light quantity distribution.

When the numerical value at each reflective surface is the result of the sum of numerical values of the pixels in the image based on inspection light, the light quantity distribution characteristic obtained based on each numerical value involves inaccuracy. Thus, it is preferable to estimate the light quantity distribution characteristic from the numerical value of each pixel.

When the light quantity distribution characteristic is estimated from the numerical value of each pixel, the aspect ratio in the X direction and the Y direction is 3:1 in the first reflective surface, and the aspect ratio in the second reflective surface is 1:1.

It is possible to calculate the light quantity using one of the following (B1), (B2), (B3), and (B4).

(B1) Sum of numerical values of pixels.
(B2) Maximum value.
(B3) Numerical value in the second or subsequent position from the maximum value.
(B4) Average of numerical values in descending order from the maximum value.

In inspection, the irradiation region is irradiated with inspection light while the irradiation position is changed. Thus, the position of the image based on inspection light changes every time an image is generated. In calculation of light quantity and luminous intensity distribution, the position of the image based on inspection light in the entire image affects the accuracy of the calculation result.

The positioning of the reflector relative to the light guide member is performed substantially accurately. In this case, the position of the image based on inspection light in the entire image substantially matches the expected position. Thus, even when the position of the image based on inspection light changes, it is possible to easily associate the image based on inspection light with the incident position information.

The positioning of the reflector relative to the distal end of the endoscope is performed substantially accurately. However, depending on the kind of endoscope, the positioning of the reflector relative to the distal end of the endoscope may be slightly inaccurate. In this case, the pixels corresponding to the image based on inspection light may be defined as a region, and the maximum value or the like in the region may be acquired.

Further, the numerical values of pixels in the image based on inspection light may be less reliable values. In this case, the numerical values are fed back to the illumination device side, so that the brightness of illumination light is adjusted. Subsequently, an image based on inspection light is generated using the adjusted illumination light, and numerical values of pixels are acquired again. Then, the acquired numerical values are divided by a value corresponding to the light source light quantity to calculate converted values. The converted values may be used to calculate light quantity and luminous intensity distribution.

A light distribution inspection method will now be described.

First Light Distribution Inspection Method

A light distribution inspection method of the present embodiment includes: generating inspection light from illumination light having a beam diameter including an incident surface of a light guide, the inspection light being incident light incident on the light guide; generating an image based on the inspection light in association with incident position information on an incident position of the inspection light; and calculating light distribution information based on the image based on the inspection light and the incident position information, the light distribution information being information on a light distribution characteristic of inspection light emitted from the light guide.

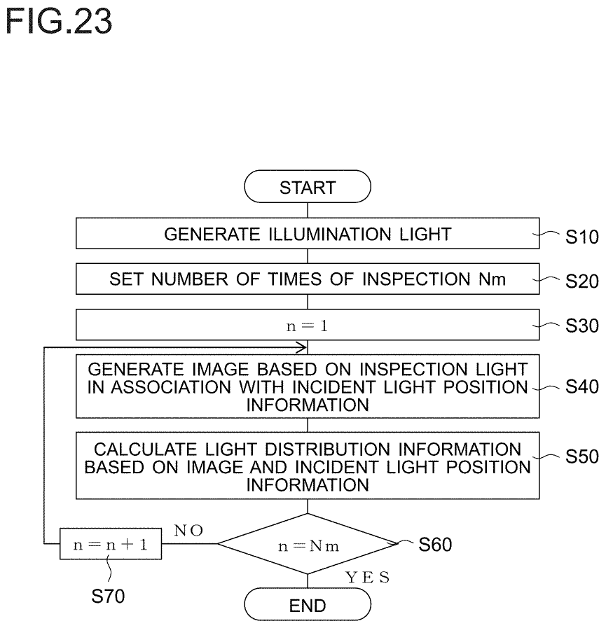

FIG. 23 is a flowchart of a first light distribution inspection method. The first light distribution inspection method includes step S10, step S20, step S30, step S40, step S50, step S60, and step S70.

At step S10, illumination light is generated.

In light distribution inspection, inspection light is used. Illumination light is generated in order to use inspection light. As illustrated in FIG. 3A and FIG. 3B, the illumination light L_{ILL} has a beam diameter including the incident surface 63 of the light guide 61.

At step S20, the number of times of inspection Nm is set.

In light distribution inspection, the incident surface 63 is repeatedly irradiated with the inspection light L_{MEA} while the irradiation position of the inspection light L_{MEA} is changed. The irradiation of the inspection light L_{MEA} is performed until the entire incident surface 63 is irradiated with the inspection light L_{MEA}.

The number of times of inspection Nm is the number of times of irradiation of the inspection light L_{MEA}. The number of times of inspection Nm can be set, for example, based on the value obtained when the area of the incident surface 63 is divided by the area of the region irradiated with the inspection light L_{MEA}.

At step S30, the value of variable n is set to 1.

At step S40, an image based on the inspection light is generated in association with incident position information.

For example, if the inspection light is picked up by the imager, it is possible to generate an image based on the inspection light. In light distribution inspection, the irradiation position of the inspection light L_{MEA} is changed relative to the incident surface 63. Every time the irradiation position is changed, an image based on the inspection light is generated.

It is possible to specify the irradiation position. The irradiation position represents the incident position of the inspection light L_{MEA}. When information on the incident position of the inspection light L_{MEA} is used as incident position information, it is possible to generate an image based on the inspection light in association with the incident position information.

At step S50, light distribution information is calculated based on the image and the incident position information.

The light distribution information is information on the light distribution characteristic of the inspection light emitted from the light guide. In the image based on the inspection light, the light quantity distribution characteristic of the inspection light L_{MEA} is imaged. The light quantity distribution characteristic includes the light distribution characteristic of the inspection light L_{MEA} emitted from the light guide 61. Thus, it is possible to calculate information on the light distribution characteristic from the image based on the inspection light.

Further, the image is associated with the incident position information. Thus, it is possible to calculate light distribution information, based on the image and the incident position information.

As described above, in the image, the light quantity distribution characteristic of the inspection light is imaged. It is possible to acquire the light quantity distribution characteristic from the image. Thus, it is possible to acquire the light quantity distribution characteristic in association with the incident position information.

At step S60, it is determined whether the value of variable n matches the number of times of inspection Nm.

If the determination result is NO, step S70 is performed. If the determination result is YES, the process ends.

If the determination is NO: n≠Nm

At step S70, 1 is added to the value of variable n. If the determination result is NO, the value of variable n does not match the number of times of inspection Nm. If the value of variable n does not match the number of times of inspection Nm, it means that the incident surface 63 is not entirely irradiated with the inspection light L_{MEA}.

If step S70 is finished, the process returns to step S40. At step S70, the value of variable n is incremented by one. Therefore, step S40 and step S50 are performed for another position of the irradiation region.

Step S40 and step S50 are repeatedly performed until the incident surface 63 is entirely irradiated with the inspection light L_{MEA}.

If the determination is YES: n=Nm

If the determination result is YES, the value of variable n matches the number of times of inspection Nm. If the value of variable n matches the number of times of inspection Nm, it means that the incident surface 63 is entirely irradiated with the inspection light L_{MEA}.

In this way, in the first light distribution inspection method, calculation of the light distribution information is performed while the irradiation position of the inspection light L_{MEA} is changed. The light distribution information is information on the light distribution characteristic of the inspection light emitted from the light guide.

In the first light distribution inspection method, it is preferable that the inspection light be part of the illumination light, an irradiation region be a region irradiated with the inspection light, the irradiation region be narrower than the incident surface, the incident position information include information on a position of the irradiation region, and generation of the image, association of the image with the incident position information, and calculation of the light distribution information be performed while the position of the irradiation region is changed.

The illumination light generated at step S10 is used for inspection light. The inspection light is part of the illumination light. Further, the irradiation region is a region irradiated with the inspection light.

As described above, in light distribution inspection, the incident surface 63 is repeatedly irradiated with the inspection light L_{MEA} while the irradiation position of the inspection light L_{MEA} is changed. Thus, as illustrated in FIG. 14A, FIG. 14B, and FIG. 14C, a region narrower than the incident surface 63 is irradiated with the inspection light L_{MEA}. Since the irradiation region is a region irradiated with the inspection light L_{MEA}, the irradiation region is narrower than the incident surface.

The incident position information is information on the incident position of the inspection light L_{MEA}. The position of the irradiation region represents the incident position of the inspection light L_{MEA}. Thus, the incident position information includes information on the position of the irradiation region.

Step S40 and step S50 are repeatedly performed, whereby generation of the image based on the inspection light, association of the image based on the inspection light with the incident position information, and calculation of the light distribution information are performed while the position of the irradiation region is changed.

Second Light Distribution Inspection Method

In the light distribution inspection method of the present embodiment, it is preferable that the light distribution information be analyzed to calculate light quantity and luminous intensity distribution, and association be performed using the incident position of the inspection light, the light quantity, and the luminous intensity distribution.

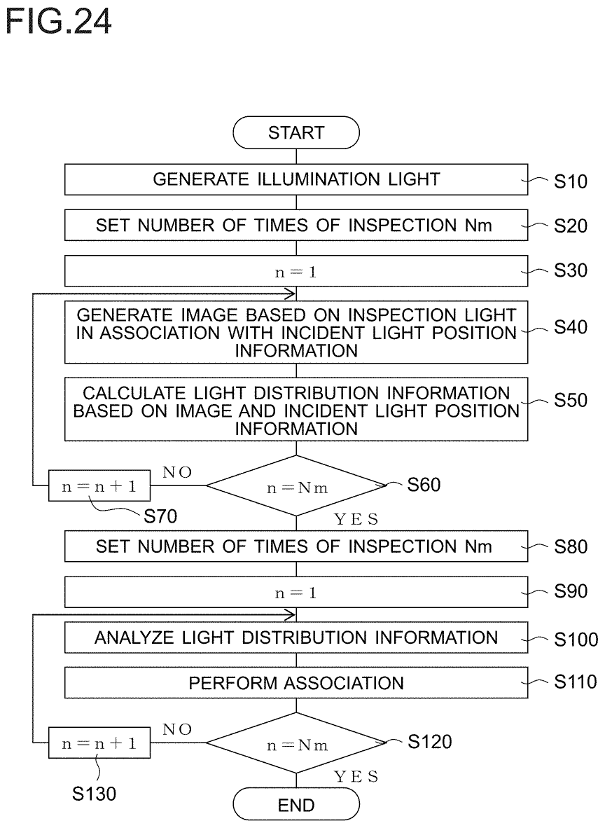

FIG. 24 is a flowchart of a second light distribution inspection method. The same steps in the first light distribution inspection method are denoted by the same numerals and a description thereof will be omitted.

The second light distribution inspection method includes the steps in the first light distribution inspection method and also includes step S80, step S90, step S100, step S110, step S120, and step S130.

At step S80, the number of times of inspection Nm is set.

In the second light distribution inspection method, a plurality of images based on inspection light are generated. In the second light distribution inspection method, the process is performed for each of the images based on the inspection light. The number of generated images based on the inspection light is equal to the number of times of inspection. Thus, it is possible to use the number of times of inspection Nm to set the number of times of process.

At step S90, the value of variable n is set to 1.

At step S100, the light distribution information is analyzed.

At step S40, an image based on the inspection light is generated. At step S50, light distribution information is calculated using the image based on the inspection light. The image based on the inspection light is formed of a plurality of pixels. By analyzing the pixels, it is possible to calculate light quantity and luminous intensity distribution.

At step S110, association is performed.

In the association, the incident position of the inspection light, the light quantity, and the luminous intensity distribution are used. The position of the irradiation region represents the incident position of the inspection light. Thus, in the association, the position of the irradiation region, the light quantity, and the luminous intensity distribution may be used.

In the first light distribution inspection method and the second light distribution inspection method, an image based on the inspection light is generated. A method of generating an image based on the inspection light will be described.

Method of Generating Image Based on Inspection Light

In the light distribution inspection method of the present embodiment, it is preferable that each of images based on the inspection light be a part of a picked-up image of a reflector having a plurality of reflective surfaces, the picked-up image of the reflector be formed of a plurality of regions, and each of the regions be a region corresponding to one of the reflective surfaces.

In the image based on the inspection light, a reflector is used. For example, it is possible to use the reflector 200 illustrated in FIG. 17B as the reflector. The reflector 200 has a plurality of reflective surfaces. By picking up an image of the reflector 200, the entire image with the reflector 200 is acquired.

As illustrated in FIG. 18B and FIG. 18D, the entire image IM_{ALL1} and the entire image IM_{ALL2} include an image based on the inspection light. In this way, the image based on the inspection light is a part of the picked-up image of the reflector.

Further, the entire image IM_{ALL1} and the entire image IM_{ALL2} are each formed of a plurality of regions. Each of the regions is a region corresponding to one of the reflective surfaces.

In the second light distribution inspection method, the light quantity is calculated. The light quantity will be described.

Method of Light Quantity

In the light distribution inspection method of the present embodiment, it is preferable that the light quantity be calculated using one of the following (a), (b), (c), and (d).

(a) Sum of numerical values of pixels.
(b) Maximum value.
(c) Numerical value in the second or subsequent position from the maximum value.
(d) Average of numerical values in descending order from the maximum value.

At step S100, the light quantity is calculated as information on the light quantity distribution characteristic of the inspection light. It is possible to calculate the light quantity using one of (B1), (B2), (B3), and (B4) as described above.

In the light distribution inspection device of the present embodiment and the light distribution inspection method of the present embodiment, light distribution information is calculated. This light distribution information can be calculated using an endoscope system. The endoscope system capable of calculating the light distribution information will be described.

An endoscope system of the present embodiment includes: a light source device including a light source and an illumination controller configured to control emission light from the light source; and an endoscope including a light guide member connectable to the light source device and having a light guide, an image pickup unit configured to acquire an image, a memory configured to store therein light distribution information of illumination light generated based on the emission light, and a plurality of emission sections optically connected to the light guide and each configured to emit a plurality of beams of illumination light based on the emission light, wherein the illumination controller controls the emission light based on the light distribution information acquired from the memory to control light distribution of the illumination light emitted from at least one of the emission sections.

The light guide has an incident surface and an emission surface. A case where the light guide has one incident surface and two emission surfaces will be described below. One of the two emission surfaces is defined as a first emission surface and the other is defined as a second emission surface.

A case where the light guide has one emission surface may be considered as using only one of the first emission surface and the second emission surface.

Endoscope System in First Example

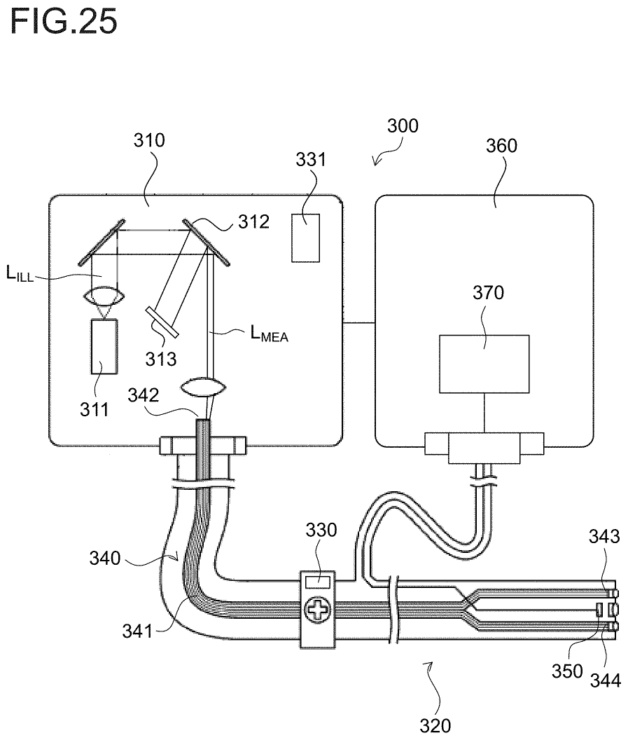

FIG. 25 is a diagram illustrating an endoscope system in a first example. An endoscope system 300 includes a light source device 310 and an endoscope 320.

The light source device 310 includes a light source 311 and an illumination controller 312. The illumination controller 312 controls emission light from the light source 311. The light source device 310 may include a light-shielding plate 313.

The endoscope 320 includes a light guide member 340, an image pickup unit 350 configured to acquire an image, a memory 330, a first emission section 343, and a second emission section 344.

The light guide member 340 is connectable to the light source device 310 and includes a light guide 341. The light guide 341 has an incident surface 342.

The memory 330 stores therein light distribution information of illumination light generated based on the emission light.

The first emission section 343 and the second emission section 344 are, for example, lenses for illumination. The first emission section 343 and the second emission section 344 are optically connected to the light guide 341. A plurality of beams of illumination light are emitted from each of the first emission section 343 and the second emission section 344 based on the emission light.

In the illumination controller 312, the emission light is controlled based on the light distribution information acquired from the memory 330. With this control, light distribution of the illumination light emitted from at least one of the emission sections is controlled.

In the endoscope system 300, at least one of light distribution of the illumination light emitted from the first emission section 343 and light distribution of the illumination light emitted from the second emission section 344 is controlled.

The endoscope system 300 may include a control device 360. The control device 360 includes, for example, an image processing device 370.

In the endoscope system 300, the light source device 310 and the endoscope 320 are separate. In this case, the endoscope system 300 can be considered as an endoscope system including a non-wireless endoscope and a light source device.

In the endoscope system 300, the light source device 310 and the endoscope 320 are connected through an adapter. Further, the image pickup unit 350 and the control device 360 are connected by a signal line.

Endoscope System in Second Example

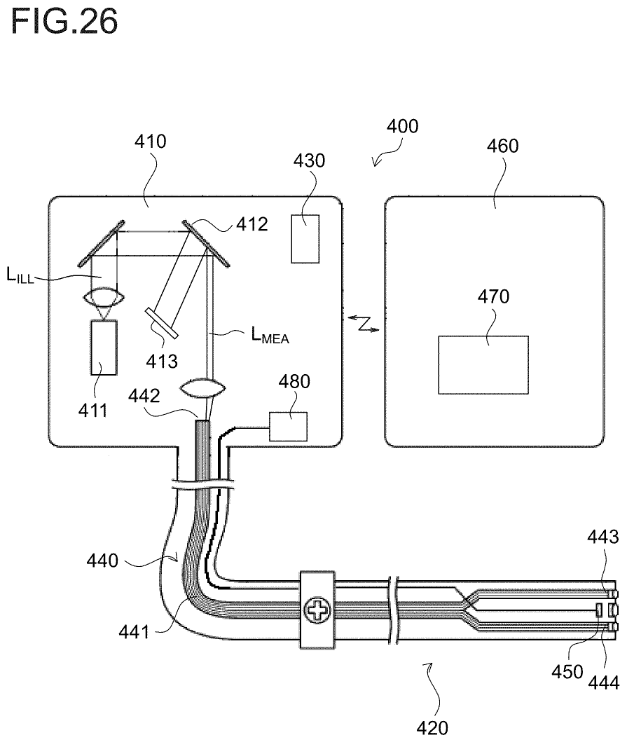

FIG. 26 is a diagram illustrating an endoscope system in a second example. An endoscope system 400 includes a light source device 410 and an endoscope 420.

The light source device 410 includes a light source 411 and an illumination controller 412. The illumination controller 412 controls emission light from the light source 411. The light source device 410 may include a light-shielding plate 413 and a communication unit 480.

The endoscope 420 includes a light guide member 440, an image pickup unit 450 configured to acquire an image, a memory 430, a first emission section 443, and a second emission section 444.

The light guide member 440 is connectable to the light source device 410 and includes a light guide 441. The light guide 441 has the incident surface 442.

The memory 430 stores therein light distribution information of illumination light generated based on the emission light.

The first emission section 443 and the second emission section 444 are, for example, lenses for illumination. The first emission section 443 and the second emission section 444 are optically connected to the light guide 441. A plurality of beams of illumination light are emitted from each of the first emission section 443 and the second emission section 444 based on the emission light.

In the illumination controller 412, the emission light is controlled based on the light distribution information acquired from the memory 430. With this control, light distribution of the illumination light emitted from at least one of the emission sections is controlled.

In the endoscope system 400, at least one of light distribution of the illumination light emitted from the first emission section 443 and light distribution of the illumination light emitted from the second emission section 444 is controlled.

The endoscope system 400 may include a control device 460. The control device 460 includes, for example, an image processing device 470.

In the endoscope system 400, the light source device 410 and an endoscope 420 are integrated. In this case, the endoscope system 400 can be considered as a wireless endoscope.

In the endoscope system 400, the light source device 410 and the endoscope 420 are connected directly. Further, an output signal from the image pickup unit 450 is transmitted wirelessly from the communication unit 480 to the control device 460.

In the endoscope system of the present embodiment, it is preferable that the endoscope system further include another memory configured to store therein correspondence information, the light distribution information be related with a manner of light distribution control in the correspondence information, and the illumination controller be controlled based on the light distribution information and the correspondence information.

As illustrated in FIG. 25, the endoscope system 300 includes the memory 331. The memory 331 stores therein the correspondence information. In the correspondence information, the light distribution information is related with a manner of light distribution control.

The light distribution information is calculated based on an image based on the inspection light L_{MEA} and the incident position information. Since the incident position information is information on the incident position of the inspection light, the light distribution information is linked with the incident position of the inspection light.

As described below, it is possible to use a digital mirror device for the illumination controller 312. In the digital mirror device, each mirror element is controlled to be in either one of the first state or the second state. Light distribution in the first state is different from light distribution in the second state.

Since light distribution in the first state is different from light distribution in the second state, it is possible to determine a manner of light distribution control with the digital mirror device.

In the digital mirror device, the position of a mirror element is linked with the incident position of inspection light. Thus, by using the digital mirror device, it is possible to link a manner of light distribution control with the incident position of inspection light.

As described above, light distribution information is linked with the incident position of inspection light. Thus, it is possible to relate light distribution information with a manner of light distribution control, using the incident position of inspection light.

In the endoscope system 300, the light source device 310 and the endoscope 320 are connected through an adapter. When the light source device 310 and the endoscope 320 are connected, the light distribution information stored in the memory 330 is read by the light source device 310. The illumination controller 312 is controlled using the read light distribution information and the correspondence information stored in the memory 331.

In the endoscope system of the present embodiment, it is preferable that the illumination light be emitted from the light source device, the inspection light be part of the illumination light, an irradiation region be a region irradiated with the inspection light, the irradiation region be narrower than the incident surface, the incident position information include information on a position of the irradiation region, and generation of the image, association of the image with the incident position information, and calculation of the light distribution information be performed while the position of the irradiation region is changed.

The inspection light will be described using the endoscope system 300. Illumination light L_{ILL} is generated in the light source device 310. The generated illumination light L_{ILL} is emitted from the light source device 310. The incident surface 342 is irradiated with part of the illumination light L_{ILL} as inspection light L_{MEA}.

The illumination light L_{ILL} has a beam diameter including the incident surface 342. At a time of light distribution inspection, the illumination light L_{ILL} is divided by the illumination controller 312 into light toward the incident surface 342 and light toward the light-shielding plate 313.

The light toward the incident surface 342 is used as the inspection light L_{MEA}. Since the inspection light L_{MEA} is part of the illumination light L_{ILL}, a region narrower than the incident surface 342 is irradiated with the inspection light L_{MEA}.

The incident surface 342 is irradiated with the inspection light L_{MEA}, whereby the inspection light L_{MEA1} is emitted from a first emission section 343 and the inspection light L_{MEA2} is emitted from a second emission section 344.

It is possible to allow the inspection light L_{MEA1} and the inspection light L_{MEA2} to be incident on the image pickup unit 350, for example, by using the reflector 200 illustrated in FIG. 17A. As a result, a first image and a second image are generated.

The first image is an image based on the inspection light L_{MEA1} emitted from the first emission section 343. The second image is an image based on the inspection light L_{MEA2} emitted from the second emission section 344

The position of the region irradiated with the inspection light L_{MEA} is acquired as incident position information. The first image is generated in association with the incident position information. Further, the second image is generated in association with the incident position information.

First light distribution information is calculated based on the first image and the incident position information. Second light distribution information is calculated based on the second image and the incident position information.

The first light distribution information is information on the light distribution characteristic of the inspection light L_{MEA1} emitted from the first emission section 343. The second light distribution information is information on the light distribution characteristic of the inspection light L_{MEA2} emitted from the second emission section 344.

In inspection, the incident surface 342 is repeatedly irradiated with the inspection light L_{MEA} while the position of the irradiation region relative to the incident surface 342 is changed. The irradiation of the inspection light L_{MEA} is performed until the entire incident surface 342 is irradiated with the inspection light L_{MEA}.

Generation of the first image, generation of the second image, association of the first image with first incident position information, association of the second image with second incident position information, calculation of the first light distribution information, and calculation of the second light distribution information are performed while a position of the region is changed. The first light distribution information and the second light distribution information are stored in the memory 330.

In FIG. 25, the memory 331 is disposed in the light source device 310. However, the memory 331 may be disposed in the control device 360.

In the endoscope system of the present embodiment, it is preferable that the illumination controller be a digital mirror device, and the light distribution information be analyzed to calculate light quantity and luminous intensity distribution.

It is possible to use a digital micromirror for the illumination controller 312 and the illumination controller 412. By using a digital micromirror for the illumination controller 312, it is possible to efficiently and easily divide the illumination light L_{ILL} into light toward the incident surface 342 and light toward the light-shielding plate 313.

As a result, in the endoscope system 300, it is possible to irradiate the incident surface 342 with the inspection light L_{MEA} repeatedly while the position of the irradiation region relative to the incident surface 342 is changed. This is applicable to the endoscope system 400.

The first light distribution information is calculated using the first image. The first image is formed of a plurality of pixels. By analyzing the pixels, it is possible to calculate first light quantity and first luminous intensity distribution.

The second light distribution information is calculated using the second image. The second image is formed of a plurality of pixels. By analyzing the pixels, it is possible to calculate second light quantity and second luminous intensity distribution.

A method of converting the position at each point of an image with inspection light into a position on an image of a subject will be described. The subject is an object picked up by the image pickup unit.

First Conversion Method

It is preferable that the endoscope system of the present embodiment include an image processing circuit, and in the image processing circuit, a position of each point in the image be converted into a position on an image of a subject, based on the image of the subject acquired by the image pickup unit.

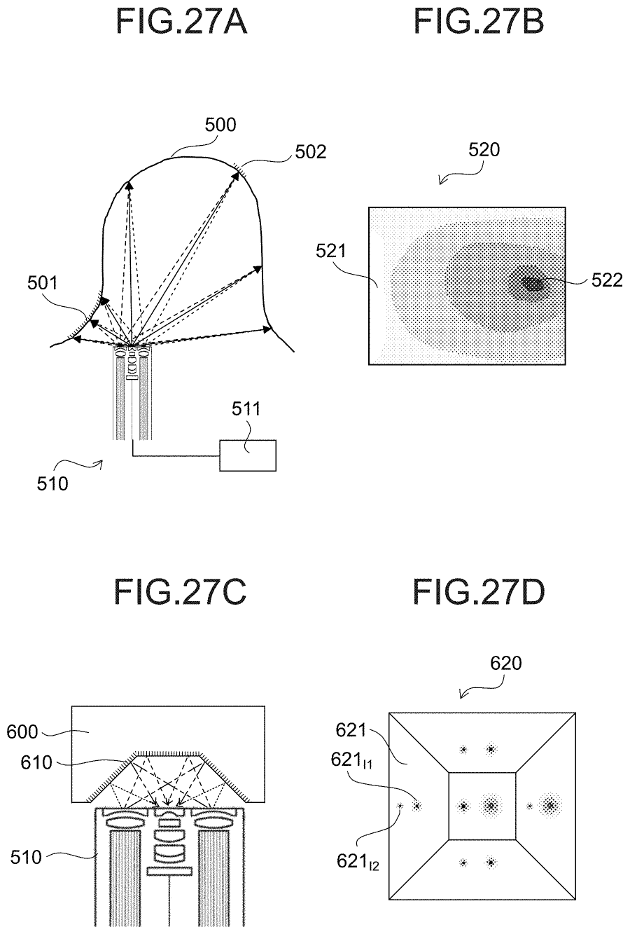

FIG. 27A, FIG. 27B, FIG. 27C, and FIG. 27D are diagrams illustrating an image pickup state and an image. FIG. 27A is a diagram illustrating image pickup of a subject. FIG. 27B is a diagram illustrating an image of the subject. FIG. 27C is a diagram illustrating image pickup of a reflector. FIG. 27D is a diagram illustrating an image of the reflector.

As illustrated in FIG. 27A, it is possible to pick up an image of a subject 500 with an endoscope system 510. As illustrated in FIG. 27B, an image 520 of the subject 500 is acquired by image pickup.

In a region 501, the distance from the endoscope system 510 to the subject 500 is short. In this case, the region 501 is illuminated very brightly. Therefore, in the image 520, an image 521 corresponding to the region 501 is a pure white image.

In a region 502, the distance from the endoscope system 510 to the subject 500 is long. In this case, the region 502 is illuminated very darkly. Therefore, in the image 520, an image 522 corresponding to the region 502 is a pure black image.

To achieve a proper contrast in the image 521, the light quantity of illumination light applied to the region 501 is reduced.

As illustrated in FIG. 27C, when an image of a reflector 600 is picked up by the endoscope system 510, an entire image 620 illustrated in FIG. 27D is acquired. The entire image 620 includes an image of a reflective region 610 based on the inspection light.

Both of the image 520 and the entire image 620 are acquired using the endoscope system 510. Thus, it can be understood that the region 501 is located in a region 621 by superimposing the image 520 and the entire image 620.

The region 621 includes a first image 621_{I1} and a second image 621_{I2}. The first image 621_{I1} and the second image 621_{I2} are generated from the same inspection light.

The position of the region irradiated with the inspection light on the incident surface of the light guide member is included in the incident position information. Then, the region at the position is prevented from being irradiated with illumination light, based on the incident position information. With this, it is possible to reduce the light quantity of illumination light applied to the region 501.

However, the shape of the recess and the depth of the recess differ between the subject 500 and the reflector 600. Further, the distance from the endoscope system 510 to the subject 500 is different from the distance from the endoscope system 510 to the reflector 600. Thus, it cannot be said that it is possible to reduce the light quantity of illumination light applied to the region 501 even when the position is prevented from being irradiated with illumination light based on the incident position information.

The endoscope system 510 includes an image processing device 511. In the image processing device 511, a corresponding point in the image 520 is found for each point in the first image 621_{I1} and each point in the second image 621_{I2}, based on the image 520. In other words, the position of each point in the first image 621_{I1} and the position of each point in the second image 621_{I2} are converted into positions in the image 520.

By doing so, where in the image 520 the first image 621_{I1} and the second image 621_{I2} are located is identified. Conversely, where in the first image 621_{I1} and the second image 621_{I2} the image 520 is located is identified.

A plurality of first images and a plurality of second images are acquired. Thus, it is possible to specify the first image and the second image located in the region 501.

For each point in the entire image 620, the corresponding point in the image 520 may be found. In other words, the position of each point in the entire image 620 may be converted to a position in the image 520. By doing so, where in the entire image 620 the region 501 is located is identified.

When the position of the region corresponding to the region 501 is identified in the entire image 620, the position of the region irradiated with inspection light on the incident surface of the light guide member is identified, from the incident position information of the image of inspection light at the position. Thus, by preventing the region at the position from being irradiated with illumination light, it is possible to reduce the light quantity of illumination light applied to the region 501.

In the endoscope system 510, the light guide member 90 is used as a light guide member. However, the light guide member 60 may be used as a light guide member. In this case, the number of images based on the inspection light is one. This can be considered as using only the first image or using only the second image in the endoscope system 510.

Second Conversion Method

It is preferable that the endoscope system of the present embodiment include an image processing circuit, in the image processing circuit, a position of each point in the image be converted into a position on an image of an object based on a subject distance pattern created in advance or a subject distance pattern at present, and the subject distance pattern at present be acquired by analyzing a plurality of images of a subject acquired by the image pickup unit.

In the endoscope system, an image of a subject or an object is picked up by the image pickup unit. An image of the subject or an image of the object is obtained by image pickup. It is possible to use the image for observation.

The subject to be observed varies with the kinds of endoscope. However, in endoscopes of the same kind, the shape of the subject is almost the same. Thus, it is possible to set a subject distance pattern created in advance and perform position conversion.

The subject distance pattern created in advance is a virtual subject. Based on the image obtained from the virtual subject, it is possible to convert the position of each point in a first image and the position of each point in a second image into positions in an image obtained from the virtual subject.

It is possible to use a dome-shaped object for the subject distance pattern created in advance. In the dome-shaped object, for example, the distance from the center of the dome to the endoscope system may be 50 mm, and the distance from the periphery of the dome to the endoscope system may be 20 mm.

In the subject distance pattern created in advance, the distance to an observation device is not actually measured but the distance to the observation device is known. Therefore, it is possible to perform position conversion based on distance information. Therefore, it is possible to accurately perform position conversion.

The subject distance pattern at present is a virtual subject. Based on the image obtained from the virtual subject, it is possible to convert the position of each point in the first image and the position of each point in the second image into positions in an image obtained from the virtual subject.

It is possible to use the endoscope system as an endoscope. In image pickup of a subject with the endoscope, the distal end of the insertion section is moved relative to the subject. A sensing device is disposed at the distal end to obtain information on movement of the distal end. It is possible to set the subject distance pattern at present based on information from the sensing device.

Further, a distance measuring device may be disposed at the distal end of the insertion section. By using the distance measuring device, it is possible to actually measure the distance to the subject.

In the subject distance pattern at present, the distance to the observation device is measured. Therefore, it is possible to perform position conversion based on distance information. Therefore, it is possible to more accurately perform position conversion.

In image pickup of a subject with the endoscope, the distal end of the insertion section is moved relative to the subject. Since image pickup is performed even while the distal end is being moved, it is possible to acquire a plurality of images of the subject. By analyzing change in the acquired images of the subject, it is possible to set the subject distance pattern at present.

The position conversion has been described above. When the position conversion is performed, it is possible to adjust the brightness of illumination light in a correction target region, based on the converted position information. The correction target region is a region that requires adjustment of the brightness of illumination light. The adjustment of the brightness of illumination light will be described.

Brightness Adjustment of Illumination Light 1

In the endoscope system of the present embodiment, it is preferable that a correction target region be extracted from an image of a subject acquired by the image pickup unit, and a region irradiated with inspection light be determined such that brightness of an image of the correction target region is predetermined brightness.

As described above, the position of each point in the first image and the position of each point in the second image are converted into positions on an image of a subject. As a result, it is possible to specify the position of the first image and the position of the second image on the image of the subject.

The first image and the second image are generated from the same inspection light. Thus, the position of the region irradiated with the inspection light on the incident surface of the light guide member is identified from the incident position information of the specified first image. By preventing the region at the position from being irradiated with illumination light, it is possible to adjust the light quantity of illumination light applied to the correction target region in the subject.

The correction target region is extracted in the image of the subject. A target value V(x,y) of the brightness of the correction target region is determined. The target value V(x,y) is set such that the brightness of the correction target region is appropriate for the brightness of the entire image of the subject.

The first image and the second image located in the correction target region are extracted. The extracted first image and second image are the images after the position conversion is performed.

Light quantity Uₚ₁(m,n) is calculated in the first image before the position conversion is performed, and light quantity Uₚ₂(m,n) is calculated in the second image. By performing position conversion, the light quantity Uₚ₁(m,n) is converted into light quantity Uₚ₁(x,y), and the light quantity Uₚ₂(m,n) is converted into light quantity Uₚ₂(x,y).

The first image and the second image are selected so that the sum of the light quantity Uₚ₁(x,y) and the light quantity Uₚ₂(x,y) matches the target value V(x,y) as much as possible. By doing so, it is possible to bring the brightness of the image in the correction target region to predetermined brightness. With the predetermined brightness, the brightness of the correction target region is appropriate for the brightness of the entire image of the subject. A plurality of first images and a plurality of second images may be selected.

The incident position information is associated with the selected first image. Based on the incident position information, the position of the region irradiated with inspection light on the incident surface of the light guide member is specified. The specified region is prevented from being irradiated with illumination light. With this, it is possible to make the brightness of the correction target region appropriate.

Brightness Adjustment of Illumination Light 2

In the endoscope system of the present embodiment, it is preferable that a region be determined from a plurality of comparative images and an image of a subject acquired by the image pickup unit, and the comparative images be images acquired in advance by changing the position of the region.

It is possible to acquire various patterns of a first image and a second image by changing the position of the region irradiated with inspection light. Light quantity and luminous intensity distribution are calculated from each of the first image and the second image. A region illuminated brightly is identified in each pattern, from the light quantity and the luminous intensity distribution.

It is possible to acquire the first image and the second image in advance. It is possible to use the first image and the second image acquired in advance, as the comparative images.

When a correction target region exists in the image of the subject acquired by the image pickup unit, it is possible to select the first image and the second image illuminated brightly in the correction target region, from the image of the subject acquired by the image pickup unit and the comparative images.

The incident position information is associated with the selected first image. Based on the incident position information, the position of the region irradiated with inspection light on the incident surface of the light guide member is specified. The specified region is prevented from being irradiated with illumination light. With this, it is possible to make the brightness of the correction target region appropriate.

By doing so, it is possible to reduce the process time since real-time calculation is substantially unnecessary.

Brightness Adjustment of Illumination Light 3

In the endoscope system of the present embodiment, it is preferable that the correction target region be divided into a plurality of correction regions, and a region be determined such that predetermined brightness in the correction region is average brightness of the correction region.

The amount of calculation is large in adjustment using the light quantity Uₚ₁(x,y), the light quantity Uₚ₂(x,y), and the target value V(x,y). Therefore, a target value V′(x,y) is set. The target value V′(x,y) is the average brightness calculated using pixels included in the correction region.

The number of correction regions is less than the number of pixels included in the correction target region. Thus, it is possible to reduce the amount of calculation.

The light quantity is also changed to the light quantity Uₚ₁′(x,y) and the light quantity Uₚ₂′(x,y), for the correction region. The light quantity Uₚ₁′(x,y) and the light quantity Uₚ₂′(x,y) are also the average light quantity calculated using a plurality of pixels.

By doing so, it is possible to alleviate calculation load in the image processing device. As a result, it is possible to achieve cost reduction of the processing circuit and acceleration of the processing.

Brightness Adjustment of Illumination Light 4

In the endoscope system of the present embodiment, it is preferable that a correction target region be extracted from an image of a subject acquired by the image pickup unit, the correction target region do not include a region formed of a pixel having a maximum value and a region formed of a pixel having a minimum value, and a region be determined such that brightness of the image of the correction target region is predetermined brightness.

In the region formed of the pixel having the maximum value, blown-out highlights occur. In the region formed of the pixel having the minimum value, crushed shadows occur. A region that does not include such regions is set as the correction target region.

When the image of the subject acquired by the image pickup unit includes the correction target region and the region formed of the pixel having the maximum value, a region is determined such that the value of an image included in the correction target region is smaller. When the image of the subject acquired by the image pickup unit includes the correction target region and the region formed of the pixel having the minimum value, a region is determined such that the value of an image included in the correction target region is larger.

By doing so, it is possible to correct only the portion that particularly requires brightness correction and therefore it is possible to perform light distribution control in a simple process. As a result, it is possible to perform the process in a short time, cheaply, and quickly.

The region formed of the pixel having the maximum value and the region formed of the pixel having the minimum value may be set as the correction target region.

In order not to give a feeling of strangeness due to brightness conversion at the boundary between the correction target region and a non-correction target region, the brightness at and near the boundary may be corrected by image processing. By doing so, the user's visual stress is alleviated.

Further, qₚ may be calculated such that {V(x,y)−Σ(Up(x,y)×qₚ)}² is minimized. In the calculation, it is possible to use the nonlinear least-squares method, the Gauss-Newton method, or the Levenberg-Marquardt method.

The case using a single light source has been described above. However, a plurality of light sources may be used. An endoscope system including a plurality of light sources will be described.

In the endoscope system of the present embodiment, it is preferable that the emission sections include a first emission section and a second emission section, first illumination light for normal observation be emitted from the first emission section and the second emission section, second illumination light for special optical observation or for use in predetermined treatment be emitted from the first emission section and the second emission section, and light distribution of the first illumination light be controlled based on light distribution information on the first illumination light acquired from the memory.

Endoscope System in Third Example

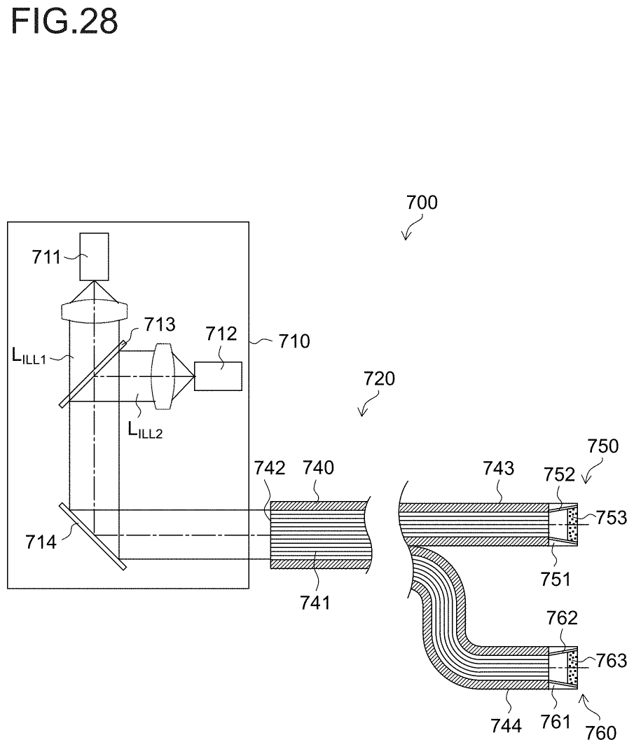

FIG. 28 is a diagram illustrating an endoscope system in a third example. An endoscope system 700 includes a light source device 710 and an endoscope 720.

The light source device 710 includes a first light source 711, a second light source 712, a dichroic mirror 713, and an illumination controller 714.

The endoscope 720 includes a light guide member 740, a first wavelength conversion unit 750, and a second wavelength conversion unit 760. The first wavelength conversion unit 750 is the first emission section, and the second wavelength conversion unit 760 is the second emission section.

The endoscope 720 includes an image pickup unit and a memory in the same manner as the endoscope 320 illustrated in FIG. 25. However, the image pickup unit and the memory are not illustrated in FIG. 28.

The light guide member 740 includes a light guide 741. On the incident end surface side, the light guide 741 has an incident surface 742. On the emission end surface side, the light guide member 740 is divided into a light guide member 743 and a light guide member 744.

Each of the light guide member 743 and the light guide member 744 has a light guide. The light guide of the light guide member 743 and the light guide of the light guide member 744 each have an emission surface.

The first wavelength conversion unit 750 is disposed on the emission surface side of the light guide member 743. The second wavelength conversion unit 760 is disposed on the emission surface side of the light guide member 744.

The first wavelength conversion unit 750 includes a first holding member 751, a first reflective member 752, and a first wavelength conversion member 753. Further, the second wavelength conversion unit 760 includes a second holding member 761, a second reflective member 762, and a second wavelength conversion member 763.

The first light source 711 and the second light source 712 are lasers. In the endoscope system 700, laser light is used as illumination light.

In the first light source 711, the wavelength of laser light L_{ILL1} is 460 nm. Thus, the laser light L_{ILL1} is blue light. In the second light source 712, the wavelength of laser light L_{ILL2} is 415 nm. Thus, the laser light L_{ILL2} is violet-blue light.

The laser light L_{ILL1} is emitted from the first light source 711. The laser light L_{ILL1} is incident on the dichroic mirror 713. The dichroic mirror 713 has optical characteristics of transmitting blue light. Thus, the laser light L_{ILL1} is transmitted through the dichroic mirror 713.

The laser light L_{ILL1} is incident on the illumination controller 714. A digital mirror device is used for the illumination controller 714. Thus, the laser light L_{ILL1} is reflected by the digital mirror device and incident on the light guide 741.

The laser light L_{ILL1} incident on the light guide 741 is emitted from the light guide member 743 and the light guide member 744. The laser light L_{ILL1} emitted from the light guide member 743 is incident on the first wavelength conversion member 753. The laser light L_{ILL1} emitted from the light guide member 744 is incident on the second wavelength conversion member 763.

The laser light L_{ILL2} is emitted from the second light source 712. The laser light L_{ILL2} is incident on the dichroic mirror 713. The dichroic mirror 713 has optical characteristics of reflecting violet-blue light. Thus, the laser light L_{ILL2} is reflected by the dichroic mirror 713.

The laser light L_{ILL2} is incident on the illumination controller 714. A digital mirror device is used for the illumination controller 714. Thus, the laser light L_{ILL2} is reflected by the digital mirror device and incident on the light guide 741.

The laser light L_{ILL2} incident on the light guide 741 is emitted from the light guide member 743 and the light guide member 744. The laser light L_{ILL2} emitted from the light guide member 743 is incident on the first wavelength conversion member 753. The laser light L_{ILL2} emitted from the light guide member 744 is incident on the second wavelength conversion member 763.

It is possible to use YAG:Ce phosphor for the first wavelength conversion member 753 and the second wavelength conversion member 763. The YAG:Ce phosphor is Ce-activated YAG phosphor. Ce represents cerium, and YAG represents yttrium, aluminum, and garnet.

Excitation light Ce is defined as excitation light in the YAG:Ce phosphor. Further, fluorescence Ce is defined as fluorescence in the YAG:Ce phosphor.

When the excitation light Ce is incident on the YAG:Ce phosphor, part of the excitation light Ce is transmitted through the YAG:Ce phosphor, and the remaining part of the excitation light Ce is absorbed by the YAG:Ce phosphor. The absorbed excitation light Ce causes fluorescence Ce to be emitted from the YAG:Ce phosphor. As a result, the excitation light Ce and the fluorescence Ce are emitted from the YAG:Ce phosphor.

Observation with White Light: Normal Observation

A case where the first light source 711 is turned on and the second light source 712 is turned off will be described. When the first light source 711 is turned on, laser light L_{ILL1} is emitted from the first light source 711. Since laser light L_{ILL2} is not emitted from the second light source 712, only the laser light L_{ILL1} is incident on the first wavelength conversion member 753 and the second wavelength conversion member 763.

As described above, the wavelength of the laser light L_{ILL1} is 460 nm. The wavelength of 460 nm is included in the wavelength range of the excitation light Ce. Therefore, the laser light L_{ILL1} acts as excitation light Ce. As a result, the laser light L_{ILL1} and the fluorescence Ce are emitted as the first illumination light from the first wavelength conversion member 753 and the second wavelength conversion member 763.

The laser light L_{ILL1} is blue light. The fluorescence Ce is yellow light. Thus, white light is emitted from the first wavelength conversion member 753 and the second wavelength conversion member 763. As a result, it is possible to perform observation with white light.

Observation with Narrow-Band Light: Special Optical Observation

A case where the first light source 711 is turned off and the second light source 712 is turned on will be described. When the second light source 712 is turned on, laser light L_{ILL2} is emitted from the second light source 712. Since laser light L_{ILL1} is not emitted from the first light source 711, only the laser light L_{ILL2} is incident on the first wavelength conversion member 753 and the second wavelength conversion member 763.

As described above, the wavelength of the laser light L_{ILL2} is 415 nm. The wavelength of 415 nm is not included in the wavelength range of the excitation light Ce. Therefore, the laser light L_{ILL2} does not act as excitation light Ce. Thus, only the laser light L_{ILL2} is emitted as the second illumination light from the first wavelength conversion member 753 and the second wavelength conversion member 763.

The laser light L_{ILL2} is blue-violet light. Thus, blue-violet light is emitted from the first wavelength conversion member 753 and the second wavelength conversion member 763. As a result, for example, it is possible to perform special optical observation with narrow-band light.

It is possible to use phosphors including YAG:Ce phosphor and SrAlO:Eu phosphor for the first wavelength conversion member 753 and the second wavelength conversion member 763. The SrAlO:Eu phosphor is Eu-activated SrAl₂O₄ phosphor. Eu represents europium, and SrAl₂O₄ represents strontium aluminate.

Excitation light Eu is defined as excitation light in the SrAlO:Eu phosphor. Further, fluorescence Eu is defined as fluorescence in the SrAlO:Eu phosphor.

When excitation light Eu is incident on the SrAlO:Eu phosphor, part of the excitation light Eu is transmitted through the SrAlO:Eu phosphor, and the remaining part of the excitation light Eu is absorbed by the SrAlO:Eu phosphor. The absorbed excitation light Eu causes fluorescence Eu to be emitted from the SrAlO:Eu phosphor. As a result, excitation light Eu and fluorescence Eu are emitted from the SrAlO:Eu phosphor.

When the first light source 711 is turned on and the second light source 712 is turned off, only the laser light L_{ILL1} is incident on the first wavelength conversion member 753 and the second wavelength conversion member 763.

As described above, the wavelength of the laser light L_{ILL1} is 460 nm. The wavelength of 460 nm is included in the wavelength range of the excitation light Ce. Therefore, the laser light L_{ILL1} acts as excitation light Ce. In comparison, the wavelength of 460 nm is not included in the wavelength range of the excitation light Eu. Therefore, the laser light L_{ILL1} does not act as excitation light Eu. As a result, the laser light L_{ILL1} and the fluorescence Ce are emitted as the first illumination light from the first wavelength conversion member 753 and the second wavelength conversion member 763.

The laser light L_{ILL1} is blue light. The fluorescence Ce is yellow light. Thus, white light is emitted from the first wavelength conversion member 753 and the second wavelength conversion member 763. As a result, it is possible to perform observation with white light.

When the second light source 712 is turned on and the first light source 711 is turned off, only the laser light L_{ILL2} is incident on the first wavelength conversion member 753 and the second wavelength conversion member 763.

As described above, the wavelength of the laser light L_{ILL2} is 415 nm. The wavelength of 415 nm is included in the wavelength range of the excitation light Eu. Therefore, the laser light L_{ILL2} acts as excitation light Eu. In comparison, the wavelength of 415 nm is not included in the wavelength range of the excitation light Ce. Therefore, the laser light L_{ILL2} does not act as excitation light Ce. As a result, the laser light L_{ILL2} and the fluorescence Eu are emitted as the second illumination light from the first wavelength conversion member 753 and the second wavelength conversion member 763.

The laser light L_{ILL2} is blue-violet light. The fluorescence Eu is green light. Thus, blue-violet light and green light are emitted from the first wavelength conversion member 753 and the second wavelength conversion member 763. Blue-violet light and green light substantially match the absorption wavelength of hemoglobin. Therefore, it is possible to observe blood vessels in high contrast.

In the endoscope system 700, light distribution of the first illumination light is controlled based on light distribution information of the first illumination light acquired from the memory. As described above, the first illumination light is white light. Thus, it is possible to make the brightness of the illumination light appropriate in observation with white light.

Endoscope System in Fourth Example

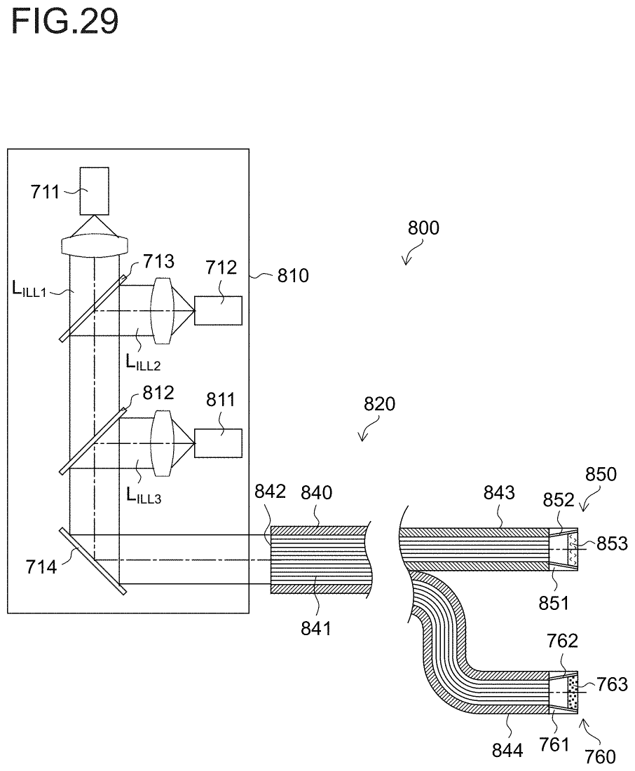

FIG. 29 is a diagram illustrating an endoscope system in a fourth example. The same configuration as that in FIG. 28 is denoted by the same number and a description thereof will be omitted.

An endoscope system 800 includes a light source device 810 and an endoscope 820.

The light source device 810 includes the first light source 711, the second light source 712, the dichroic mirror 713, the illumination controller 714, a third light source 811, and a dichroic mirror 812.

The endoscope 820 includes a light guide member 840, a third wavelength conversion unit 850, and the second wavelength conversion unit 760. The third wavelength conversion unit 850 is the first emission section, and the second wavelength conversion unit 760 is the second emission section.

The endoscope 820 includes an image pickup unit and a memory in the same manner as the endoscope 320 illustrated in FIG. 25. However, the image pickup unit and the memory are not illustrated in FIG. 29.

The light guide member 840 includes a light guide 841. On the incident end surface side, the light guide 841 has an incident surface 842. On the emission end surface side, the light guide member 840 is divided into a light guide member 843 and a light guide member 844.

Each of the light guide member 843 and the light guide member 844 has a light guide. The light guide of the light guide member 843 and the light guide of the light guide member 844 each have an emission surface.

The third wavelength conversion unit 850 is disposed on the emission surface side of the light guide member 843. The second wavelength conversion unit 760 is disposed on the emission surface side of the light guide member 844.

The third wavelength conversion unit 850 includes a third holding member 851, a third reflective member 852, and a third wavelength conversion member 853. Further, the second wavelength conversion unit 760 includes the second holding member 761, the second reflective member 762, and the second wavelength conversion member 763.

The third light source 811 is a YAG laser. In the third light source 811, the wavelength of laser light L_{ILL3} is 1064 nm. The laser light L_{ILL3} is not used for illumination.

The laser light L_{ILL3} is emitted from the third light source 811. The laser light L_{ILL3} is incident on the dichroic mirror 812. The dichroic mirror 812 has optical characteristics of reflecting infrared light. Thus, the laser light L_{ILL3} is reflected by the dichroic mirror 812.

The dichroic mirror 812 has optical characteristics of transmitting light with a wavelength shorter than the wavelength of infrared light. Thus, the laser light L_{ILL1} and the laser light L_{ILL2} are transmitted through the dichroic mirror 812.

The laser light L_{ILL3} is incident on the illumination controller 714. A digital mirror device is used for the illumination controller 714. Thus, the laser light L_{ILL3} is reflected by the digital mirror device and incident on the light guide 841.

It is possible to freely set the incident range of the laser light L_{ILL3} on the incident surface 842 by controlling the digital mirror device. Thus, it is possible to emit the laser light L_{ILL3} only from the light guide member 843.

The laser light L_{ILL3} emitted from the light guide member 843 is incident on the third wavelength conversion member 853. It is possible to use YAG:Ce phosphor for the third wavelength conversion member 853.

A case where the first light source 711 is turned on and the second light source 712 and the third light source 811 are turned off will be described. When the first light source 711 is turned on, the laser light L_{ILL1} is incident on the third wavelength conversion member 853 and the second wavelength conversion member 763.

YAG:Ce phosphor is used for the third wavelength conversion member 853 and the second wavelength conversion member 763. Therefore, the laser light L_{ILL1} and the fluorescence Ce are emitted as the first illumination light from the third wavelength conversion member 853 and the second wavelength conversion member 763. As a result, it is possible to perform observation with white light.

A case where the third light source 811 is turned on and the first light source 711 and the second light source 712 are turned off will be described. When the third light source 811 is turned on, the laser light L_{ILL3} is incident on the third wavelength conversion member 853 and the second wavelength conversion member 763.

As described above, the wavelength of the laser light L_{ILL3} is 1064 nm. The wavelength of 1064 nm is not included in the wavelength range of the excitation light Ce. Therefore, the laser light L_{ILL3} does not act as excitation light Ce. Thus, only the laser light L_{ILL3} is emitted as the second illumination light from the third wavelength conversion member 853 and the second wavelength conversion member 763.

The laser light L_{ILL3} is laser light emitted from the YAG laser. The laser light emitted from the YAG laser is used for treatment of the subject, such as cauterization, coagulation, and transpiration (hereinafter referred to as “predetermined treatment”). Thus, it is possible to use the laser light L_{ILL3} for the predetermined treatment.

To perform the predetermined treatment, a treatment site has to be irradiated with high-energy laser light L_{ILL3}. The laser light L_{ILL3} is emitted from the third wavelength conversion member 853. At this moment, if the laser light L_{ILL3} is widely diffused by the third wavelength conversion member 853, it is impossible to ensure high energy.

The third wavelength conversion member 853 includes fluorescent particles and diffusive particles. The second wavelength conversion member 763 also includes fluorescent particles and diffusive particles. In the endoscope system 800, at least one of the density of fluorescent particles and the density of diffusive particles differs between the third wavelength conversion member 853 and the second wavelength conversion member 763. Therefore, the degree of light diffusion differs between the third wavelength conversion member 853 and the second wavelength conversion member 763.

Specifically, the degree of light diffusion in the third wavelength conversion member 853 is considerably smaller than the degree of light diffusion in the second wavelength conversion member 763. Therefore, the laser light L_{ILL3} emitted from the third wavelength conversion member 853 is not diffused much even though it is transmitted through the third wavelength conversion member 853. As a result, it is possible to perform the predetermined treatment using the laser light L_{ILL3}.

In the endoscope system 800, the laser light L_{ILL3} is incident on the entire surface of the digital mirror device. However, the laser light L_{ILL3} may be incident on a limited range. The limited range is a range that allows the laser light L_{ILL3} to be emitted only from the light guide member 843.

In the endoscope system 800, light distribution of the first illumination light is controlled based on light distribution information of the first illumination light acquired from the memory. As described above, the first illumination light is white light. Thus, it is possible to make the brightness of the illumination light appropriate, for example, in normal observation with white light.

Reflector in Sixth Example

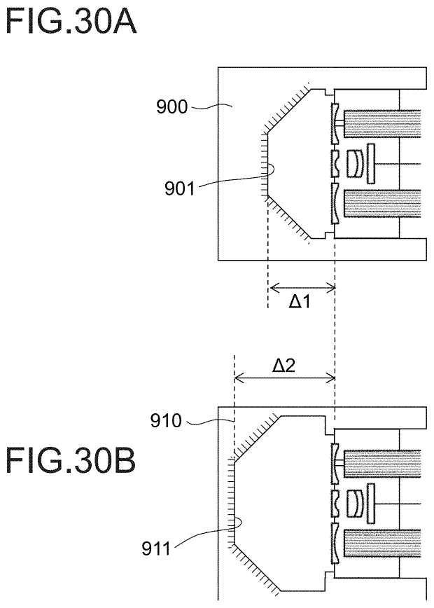

FIG. 30A and FIG. 30B are diagrams illustrating a reflector in a sixth example. FIG. 30A is a diagram illustrating a reflector of a first type. FIG. 30B is a diagram illustrating a reflector of a second type.

In the light distribution inspection device and the endoscope system, the subject is irradiated with illumination light from a plurality of directions. The position of the center of illumination light varies with the distance to the subject. Therefore, for example, when the subject is irradiated with illumination light from two directions, the distance between the center of one illumination light and the center of the other illumination light varies with the distance to the subject.

An image is acquired in a state in which the subject is irradiated with illumination light from a plurality of directions. Thus, in the image, the distance between the center of the one illumination light and the center of the other illumination light also varies with the distance to the subject. Further, in the image, the size of the subject also varies with the distance to the subject.

When the distance to the subject changes, the light quantity distribution characteristic of the illumination light on the subject changes in both of the one illumination light and the other illumination light. This is applicable also when the size of the subject changes. Change in light quantity distribution characteristic also occurs in the image. Thus, it is preferable to acquire light distribution information in accordance with the distance to the subject in order to provide illumination with light distribution adjusted according to the subject.

It is possible to acquire the light distribution information according to the distance to the subject, for example, using a reflector 900 and a reflector 910.

As illustrated in FIG. 30A, the reflector 900 has a reflective surface 901. The reflector 900 has a tilt surface around the reflective surface 901. The angle of the tilt surface is 45 degrees relative to the normal to the reflective surface 901. In the reflector 900, the reflective surface 901 and the tilt surface form a reflective region. Distance Δ1 is the distance from the endoscope distal end to the reflective surface 901.

As illustrated in FIG. 30B, the reflector 910 has a reflective surface 911. The reflector 910 has a tilt surface around the reflective surface 911. The angle of the tilt surface is 45 degrees relative to the normal to the reflective surface 911. In the reflector 910, the reflective surface 911 and the tilt surface form a reflective region. Distance Δ2 is the distance from the endoscope distal end to the reflective surface 911.

The angle of the tilt surface in the reflector 900 and the angle of the tilt surface in the reflector 910 are the same. However, the angle of the tilt surface in the reflector 900 and the angle of the tilt surface in the reflector 910 may differ.

The distance Δ2 is longer than the distance Δ1. The distance Δ2 and the distance Δ1 can be considered as the distance from the endoscope distal end to the subject. Thus, by using the reflector 900 and the reflector 910, it is possible to acquire light distribution information according to the distance to the subject.

The light distribution information acquired using the reflector 900 and the light distribution information acquired using the reflector 910 are stored in the memory. As a result, it is possible to control emission light based on the light distribution information acquired from the memory.

It is preferable that the endoscope system of the present embodiment further include a distance meter configured to detect a distance to a subject, and the illumination controller change light distribution of the illumination light in accordance with the distance to the subject, using, of the light distribution information, light distribution information corresponding to the detected distance to the subject.

To acquire light distribution information from the memory, information on the distance to the subject is required. Therefore, the endoscope system includes the distance meter configured to detect the distance to the subject.

For example, when the illumination light quantity is large but the brightness of the subject in the image is low, the distance to the subject is long. Thus, it is possible to detect the distance to the subject, based on the relation between the illumination light quantity and the brightness of the image.

The memory stores therein a plurality of pieces of light distribution information. The distance to the subject varies among the pieces of light distribution information. If the distance to the subject can be detected, light distribution information corresponding to the distance to the subject can be obtained from the memory. As a result, light distribution of illumination light can be changed by the illumination controller in accordance with the distance to the subject, using, among the pieces of light distribution information, the light distribution information corresponding to the detected distance to the subject.

A storage medium of the present embodiment stores therein a program to cause processing including: generating inspection light from illumination light having a beam diameter including an incident surface of a light guide, the inspection light being incident light incident on the light guide; generating an image based on the inspection light in association with incident position information on an incident position of the inspection light; and calculating light distribution information based on the image based on the inspection light and the incident position information, the light distribution information being information on a light distribution characteristic of the inspection light emitted from the light guide.

It is possible to use the reflector described above as a jig for light distribution inspection.

The present embodiment includes the following embodiment.

An endoscope system including:

an illumination unit including a light source and an illumination controller;

an insertion section including a light guide member including a light guide and an image pickup unit configured to acquire an image; and

a memory configured to store therein light distribution information, wherein

the light guide at least has an incident surface, a first emission surface, and a second emission surface,

inspection light is generated from illumination light having a beam diameter including the incident surface of the light guide,

the inspection light is incident light incident on the light guide,

a first image based on the inspection light emitted from the first emission surface is generated in association with incident position information on an incident position of the inspection light,

a second image based on the inspection light emitted from the second emission surface is generated in association with incident position information on an incident position of the inspection light,

first light distribution information is calculated based on the first image and the incident position information,

second light distribution information is calculated based on the second image and the incident position information, and

the first light distribution information and the second light distribution information are information on light distribution characteristic of the inspection light emitted from the light guide.

The endoscope system, wherein

illumination light is emitted from the illumination unit,

the inspection light is part of the illumination light and applied to a region narrower than the incident surface,

the incident position information includes information on a position of a region, and

generation of the first image, generation of the second image, association of the first image with the incident position information, association of the second image with the incident position information, calculation of the first light distribution information, and calculation of the second light distribution information are performed while the position of the region is changed.

The present disclosure is suitable for a light distribution inspection device and a light distribution inspection method capable of accurately and easily inspecting the light quantity and the luminous intensity distribution of light emitted from a light guide member.

The present disclosure is suitable for an endoscope system capable of providing illumination with light distribution adjusted according to the subject.

The present disclosure is suitable for a storage medium storing therein a program capable of accurately and easily inspecting the light quantity and the luminous intensity distribution of light emitted from a light guide member.

According to the present disclosure, it is possible to provide a light distribution inspection device and a light distribution inspection method capable of accurately and easily inspecting the light quantity and the luminous intensity distribution of light emitted from a light guide member.

According to the present disclosure, it is possible to provide an endoscope system capable of providing illumination with light distribution adjusted according to the subject.

According to the present disclosure, it is possible to provide a storage medium storing therein a program capable of accurately and easily inspecting the light quantity and the luminous intensity distribution of light emitted from a light guide member.

What is claimed is:

1. A light distribution inspection device comprising a processor, wherein
the processor is configured to:
acquire, based on a plurality of beams of inspection light each incident on a light guide of an illumination device, a light quantity distribution characteristic of each of a plurality of beams of emission light emitted from each of a plurality of emission sections of the illumination device optically connected to the light guide, in association with incident position information of each of the beams of inspection light;
calculate light distribution information of the illumination device, based on each of pieces of the incident position information and each of the light quantity distribution characteristics; and
analyze the light distribution information to calculate light quantity and luminous intensity distribution.

2. The light distribution inspection device according to claim 1, wherein
the inspection light is part of illumination light,
an irradiation region is a region irradiated with the inspection light,
the irradiation region is narrower than an incident surface of the light guide,
the incident position information includes information on a position of the irradiation region, and
acquisition of the light quantity distribution characteristic and calculation of the light distribution information are performed while a position of the irradiation region is changed.

3. The light distribution inspection device according to claim 1, further comprising an imager configured to output a signal to be used to generate an image, wherein
the imager faces a holding member.

4. The light distribution inspection device according to claim 1, further comprising an imager configured to output signals to be used to generate images, wherein
each of the images is a picked-up image of the inspection light reflected by a reflector, and
a holding member and the imager are disposed on one side of the reflector.

5. The light distribution inspection device according to claim 4, wherein
the reflector has a recess,
an inner surface of the recess has a reflective region formed of a plurality of reflective surfaces, and
the reflective region has a largeness that allows inspection light emitted from the light guide to be incident on the reflective region.

6. The light distribution inspection device according to claim 5, wherein
each of the reflective surfaces is planar, and
a normal to the reflective surface is inclined in each of the reflective surfaces.

7. A light distribution inspection method comprising:
generating inspection light from illumination light having a beam diameter including an incident surface of a light guide,
the inspection light being incident light incident on the light guide;
generating an image based on the inspection light in association with incident position information on an incident position of the inspection light;
calculating light distribution information based on the image based on the inspection light and the incident position information; and
analyzing the light distribution information to calculate light quantity and luminous intensity distribution,
the light distribution information being information on a light distribution characteristic of the inspection light emitted from the light guide.

8. The light distribution inspection method according to claim 7, wherein
the inspection light is part of the illumination light,
an irradiation region is a region irradiated with the inspection light,
the irradiation region is narrower than the incident surface,
the incident position information includes information on a position of the irradiation region, and
generation of the image, association of the image with the incident position information, and calculation of the light distribution information are performed while a position of the irradiation region is changed.

9. The light distribution inspection method according to claim 7, wherein
the light distribution information is analyzed to calculate light quantity and luminous intensity distribution, and
association is performed using the incident position of the inspection light, the light quantity, and the luminous intensity distribution.

10. The light distribution inspection method according to claim 7, wherein
each of images based on the inspection light is a part of a picked-up image of a reflector having a plurality of reflective surfaces,
the picked-up image of the reflector is formed of a plurality of regions, and
each of the regions is a region corresponding to one of the reflective surfaces.

11. The light distribution inspection method according to claim 10, wherein the light quantity is calculated using one of (a), (b), (C), and (d) below:
(a) Sum of numerical values of pixels,
(b) a maximum value,
(C) a numerical value in a second or subsequent position from the maximum value, and
(d) Average of numerical values in descending order from the maximum value.

12. An endoscope system comprising:
a light source device including a light source and an illumination controller configured to control emission light from the light source; and
an endoscope including a light guide member connectable to the light source device and having a light guide, an image pickup unit configured to acquire an image, a memory configured to store therein light distribution information of illumination light generated based on the emission light, and a plurality of emission sections optically connected to the light guide and each configured to emit a plurality of beams of illumination light based on the emission light, wherein
the illumination controller controls the emission light based on the light distribution information acquired from the memory to control light distribution of the illumination light emitted from at least one of the emission sections.

13. The endoscope system according to claim 12, further comprising another memory configured to store therein correspondence information, wherein
the light distribution information is related with a manner of light distribution control, in the correspondence information, and
the illumination controller is controlled based on the light distribution information and the correspondence information.

14. The endoscope system according to claim 12, wherein
the illumination light is emitted from the light source device,
an inspection light is part of the illumination light,
an irradiation region is a region irradiated with the inspection light,
the irradiation region is narrower than an incident surface,
the incident position information includes information on a position of the irradiation region, and
generation of the image, association of the image with the incident position information, and calculation of the light distribution information are performed while a position of the irradiation region is changed.

15. The endoscope system according to claim 12, wherein
the illumination controller is a digital mirror device, and
the light distribution information is analyzed to calculate light quantity and luminous intensity distribution.

16. The endoscope system according to claim 12, further comprising an image processing circuit,
wherein in the image processing circuit, a position of each point in the image is converted into a position on an image of a subject, based on the image of the subject acquired by the image pickup unit.

17. The endoscope system according to claim 12, further comprising an image processing circuit, wherein
in the image processing circuit, a position of each point in the image is converted into a position on an image of an object, based on a subject distance pattern created in advance or a subject distance pattern at present, and
the subject distance pattern at present is acquired by analyzing a plurality of images of a subject acquired by the image pickup unit.

18. The endoscope system according to claim 12, wherein
a correction target region is extracted from an image of a subject acquired by the image pickup unit, and
a region irradiated with an inspection light is determined such that brightness of an image of the correction target region is predetermined brightness.

19. The endoscope system according to claim 12, wherein
the emission sections include a first emission section and a second emission section,
first illumination light for normal observation is emitted from the first emission section and the second emission section,
second illumination light for special optical observation or for use in predetermined treatment is emitted from the first emission section and the second emission section, and
light distribution of the first illumination light is controlled based on light distribution information on the first illumination light acquired from the memory.

20. The endoscope system according to claim 12, further comprising a distance meter configured to detect a distance to a subject,
wherein the illumination controller changes light distribution of the illumination light in accordance with the distance to the subject, using, of the light distribution information, light distribution information corresponding to the detected distance to the subject.

21. A storage medium storing therein a program to cause processing comprising:
generating inspection light from illumination light having a beam diameter including an incident surface of a light guide, the inspection light being incident light incident on the light guide;
generating an image based on the inspection light in association with incident position information on an incident position of the inspection light;
calculating light distribution information based on the image based on the inspection light and the incident position information; and
analyzing the light distribution information to calculate light quantity and luminous intensity distribution,
the light distribution information being information on a light distribution characteristic of the inspection light emitted from the light guide.

22. An endoscope system comprising:
an endoscope; and
a light source device, wherein
the light source device includes
a light source an
an illumination controller configured to control emission light from the light source,
the endoscope includes
a light guide member having an incident surface on which the emission light controlled is incident,
an emission section optically connected to the light guide member and emitting light guided by the light guide member as illumination light, and
a memory configured to store therein incident position information that is a position at which the emission light is incident on the incident surface, in association with light quantity and luminous intensity distribution of the illumination light emitted from the emission section, and
the illumination controller controls the emission light based on the incident position information, the light quantity, and the luminous intensity distribution stored in the memory.
